(19) United States
(12) Patent Application Publication
(10) Pub. No.: US 2024/0148880 A1
(43) Pub. Date: May 9, 2024

(54) REJUVENATION OF CAR T CELL

(71) Applicant: Purdue Research Foundation, West Lafayette, IN (US)

(72) Inventors: Philip Stewart LOW, West Lafayette, IN (US); Boning Zhang, West Lafayette, IN (US); John V. Napoleon, West Lafayette, IN (US)

(21) Appl. No.: 18/392,939

(22) Filed: Dec. 21, 2023

Related U.S. Application Data
(63) Continuation of application No. 17/266,509, filed on Feb. 5, 2021.
(60) Provisional application No. 62/715,666, filed on Aug. 7, 2018.

(51) Int. Cl. A61K 47/55; A61K 31/437; A61K 31/4535; A61K 31/4545; A61K 31/7076; A61K 47/62; A61K 47/68; A61K 49/00

(52) U.S. Cl. CPC A61K 47/55; A61K 31/437; A61K 31/4535; A61K 31/4545; A61K 31/7076; A61K 47/62; A61K 47/6835; A61K 49/0043; A61K 49/0052

(57) ABSTRACT

A payload of drug conjugated to a targeting ligand specifically designed to deliver to exhausted CAR T cells to rejuvenate these CAR T cells is provided herein. The targeted CAR T cells are modified with a fusion receptor which can bind to the targeting ligand and internalize the conjugated payload of drug to execute its regulatory function to exhausted CAR T cell.

### RELATED APPLICATIONS

This application is a continuation of U.S. application Ser. No. 17/266,509, filed on 5 Feb. 2021, which is a U.S. national stage filing under 35 U.S.C. 371 from International Application No. PCT/US2019/042726, filed on 21 Jul. 2019, and published as WO 2020/033129 on 13 Feb. 2020, which claims the benefit of U.S. Provisional Application Ser. No. 62/715,666, filed 7 Aug. 2018, the contents of which are specifically incorporated herein by reference in their entirety.

### FIELD OF INVENTION

This disclosure provides a system to rejuvenate cancer antigen exhausted chimeric antigen receptor T (CAR T) cells. Specifically, the system comprising a fusion receptor in a classical CAR construct, wherein the fusion receptor provides a ligand binding module that recognizes a high affinity ligand-payload drug conjugate to deliver payload of drugs that are designed to either block the inhibitory signaling in the exhausted CAR T, or to re-activate CAR T through an antigen independent pathway.

### BACKGROUND

The field of chimeric antigen receptor (CAR) T cell therapy has made tremendous progress over the last two decades. The CAR construct consists of four parts: (1) an extracellular binding moiety against tumor specific antigen, (2) a hinge domain, (3) a transmembrane domain and (4) a combination of various activation domains, for example, CD28, 4-1BB and CD3ζ chain. The most impressive successes have been seen in CAR T therapy against CD19 positive B cell leukemias, where more than 80% complete remission rate has been achieved in several clinical trials.

In contrast, the treatment of solid tumor by CAR T has so far proven to be more challenging. Great successes have been achieved in preclinical study using syngeneic mice model or xenograft tumor model in immunodeficient mice. However, none of the clinical trials involving solid tumor, for example, breast cancer, ovarian cancer or lung cancer has shown improvement compare to control. In general, it suffers from the same limitations that are faced by other adoptive cell therapies, including (1) poor tumor penetration; (2) hypoxic pressure; (3) are immunosuppressive tumor microenviroment which includes tumor associated macrophages, fibroblasts and suppressive cytokines¹. CAR T cells, as same as tumor infiltrated lymphocytes (TIL), can be re-educated by this suppressive microenviroment and turn to a “hypofunctional” status, which is characterized by overexpression of co-inhibitory molecules (i.e., PD-1, Tim-3, LAG-3 etc.) decreased INF_{γ} secretion and killing capability. Data from the patient samples of ovarian cancer shows that the majority of PD-1⁺CD8⁺ T cell lacked expression of CD127 which is known to be important for the effector-to-memory transition in T cell. Overexpression of LAG-3 also negatively correlated with the effector function of TCR specific CD8⁺ TIL. In addition, PD-1⁺ LAG-3⁺ double positive T cell exhibited lower INF_{γ} production². Similarly, NY-ESO-1 TCR specific human T cell became hypofunctional in mice solid tumor model, showing high expression of the co-inhibitory molecules and less efficient anti-tumor effect, due to both the microenvrioment and the constant activation of T cell by the continuous exposure to antigen³. Therefore, a reversion of the suppressive microenviroment, more importantly, a rejuvenation of the exhausted CAR T cell is highly desired for a better solid tumor treatment.

### SUMMARY OF THE INVENTION

This disclosure provides system to rejuvenate an exhausted classical CAR T cell. The system comprises at least two components: a first component is a conjugate comprising a targeting ligand covalently linked to a payload of drug; and a second component is a targeting ligand binding module linked to membrane-anchoring module. The targeting ligand binding module of the second component recognizes the targeting ligand in the first component with high affinity to form a complex, and the payload drug either blocks the inhibitory signaling of the exhausted CAR T, or re-activates said CAR T through an antigen independent pathway. The membrane-anchoring module mediates internalization of the two component complex into the exhausted CAR T cell.

In some preferred embodiment, the aforementioned targeting ligand of the first component is folate, FITC or FK506.

In some preferred embodiment, the aforementioned targeting ligand binding module of the second component comprises a folate receptor, an anti-FITC antibody fragment or FKBP.

In some preferred embodiment, the aforementioned membrane-anchoring module is a folate receptor.

In some preferred embodiment, the aforementioned first component comprises a releasable linker between the targeting ligand and the payload drug.

In some preferred embodiment, the aforementioned first component comprises a non-releasable linker between the targeting ligand and the payload drug.

In some preferred embodiment, the binding affinity between aforementioned targeting ligand and the targeting ligand-binding module is in sub-nanomolar range.

In some preferred embodiment, the aforementioned payload of drug is a Toll Like Receptor 7 (TLR7) agonist or Simulator of interferon genes (STING) agonist.

In some preferred embodiment, the aforementioned payload of drug is an inhibitor to following proteins: SHP1/2, TC-PTP or DGKα, TGFβ.

In some preferred embodiment, the aforementioned TLR7 agonist has the structure of or

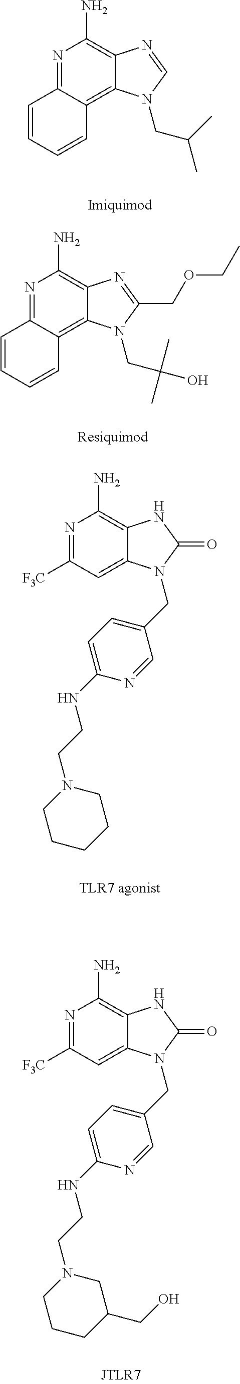 [SMILES: CC(C)Cn1cnc2c(N)nc3ccccc3c21 ; CCOCc1nc2c(N)nc3ccccc3c2n1CC(C)(C)O ; Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

In some preferred embodiment, the aforementioned first component is a Fluorescein-TLR7 agonist having the structure of

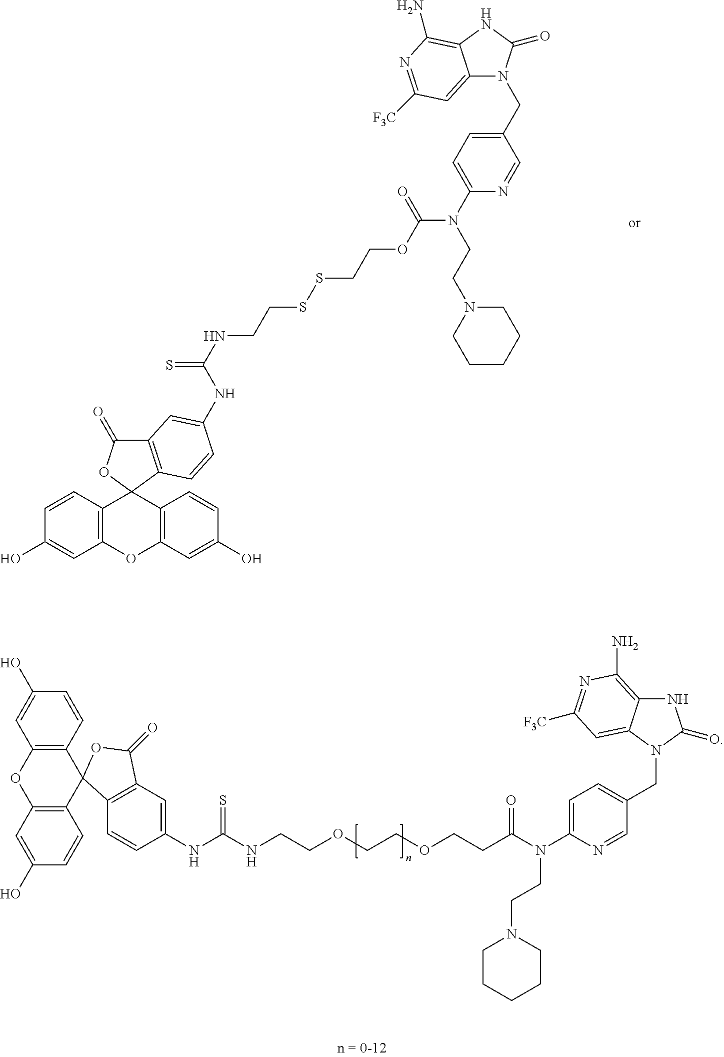

In some preferred embodiment, the aforementioned first component is a FK506-TLR7 agonist having the structure of

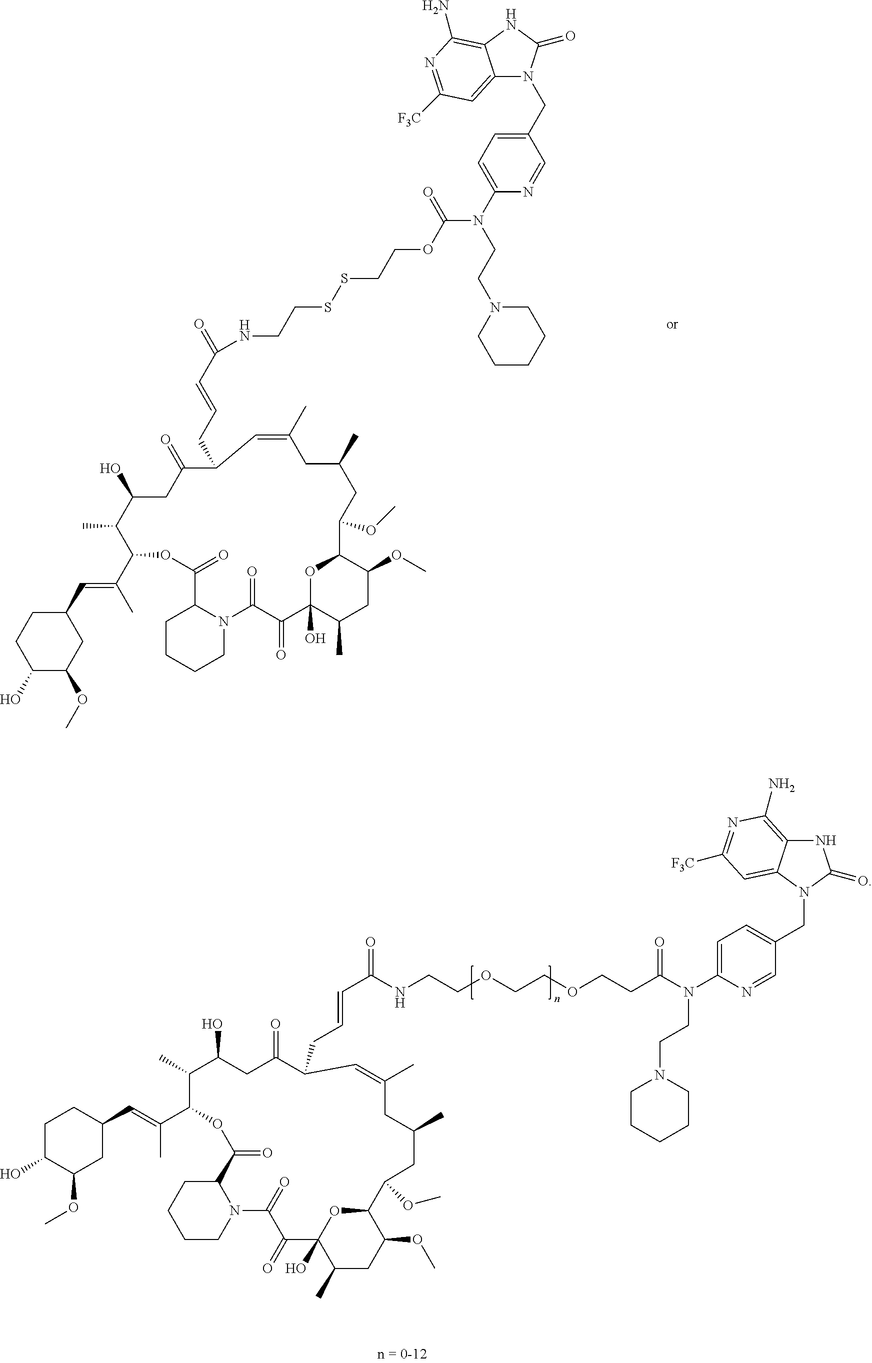

In some preferred embodiment, the aforementioned first component is one of the following:

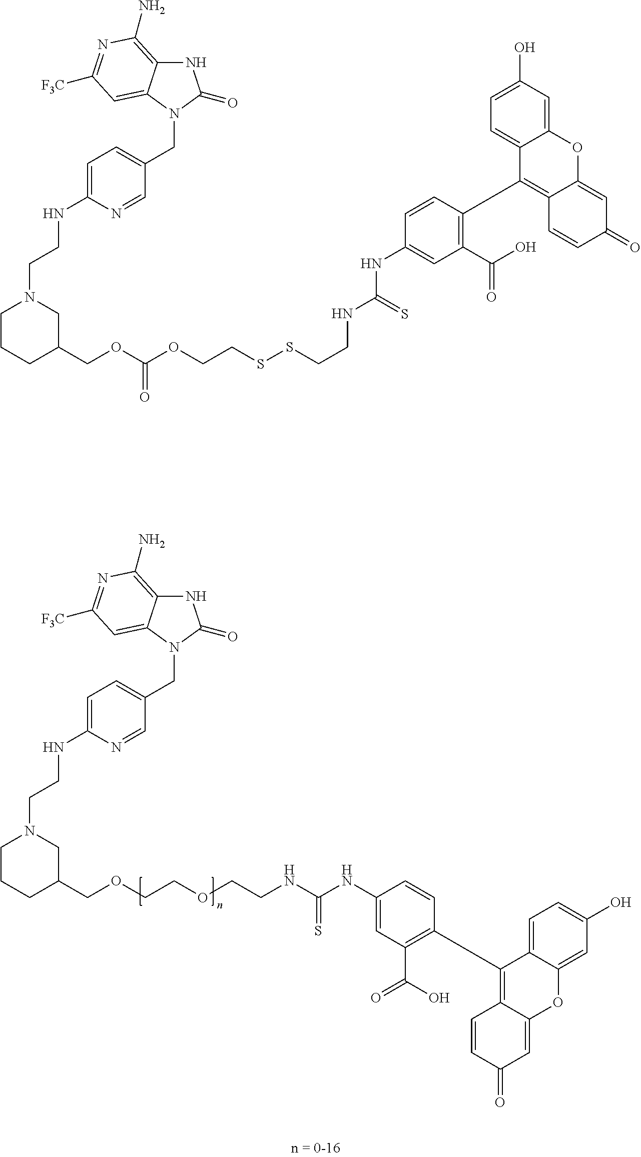

In some preferred embodiment, the aforementioned first component comprising the payload drug selected from the group consisting of following TC-PTP phosphatase inhibitors:

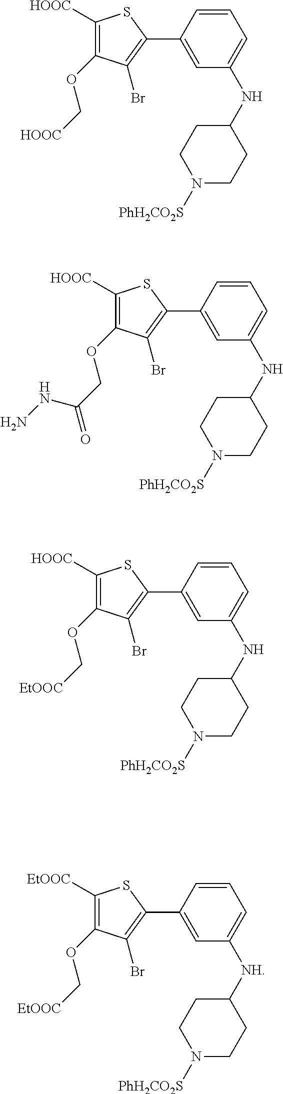

In some preferred embodiment, the aforementioned phosphatase inhibitor is connected to the fluorescein or FK506 (tacrolimus) to form the following structures:

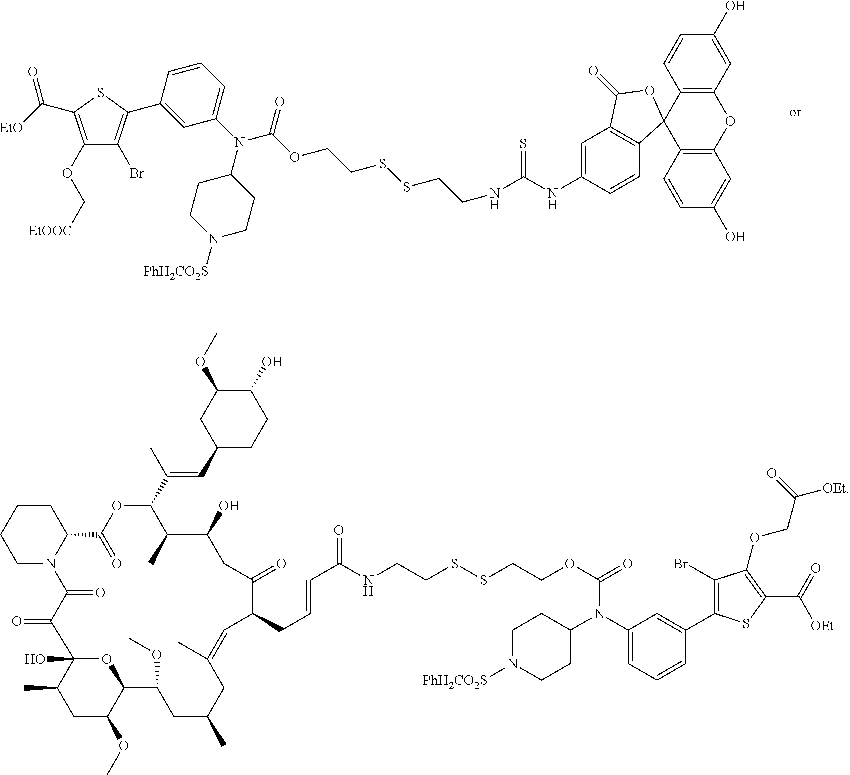

In some preferred embodiment, the aforementioned payload drug in the first component comprises a STING agonist of one of the following structures.

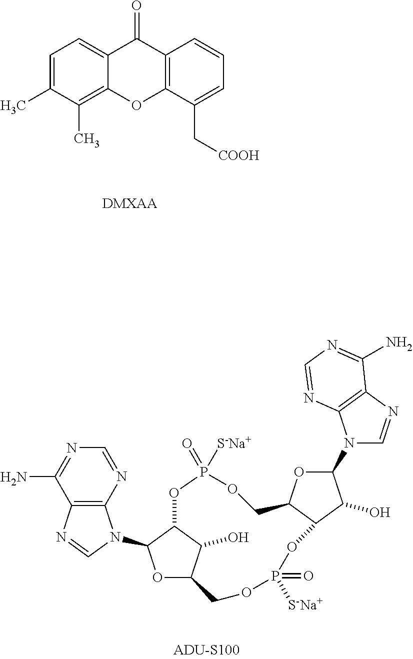

In some preferred embodiment, the aforementioned first component comprises a spacer between the targeting ligand and the payload drug selected from the group consisting of the following structures:

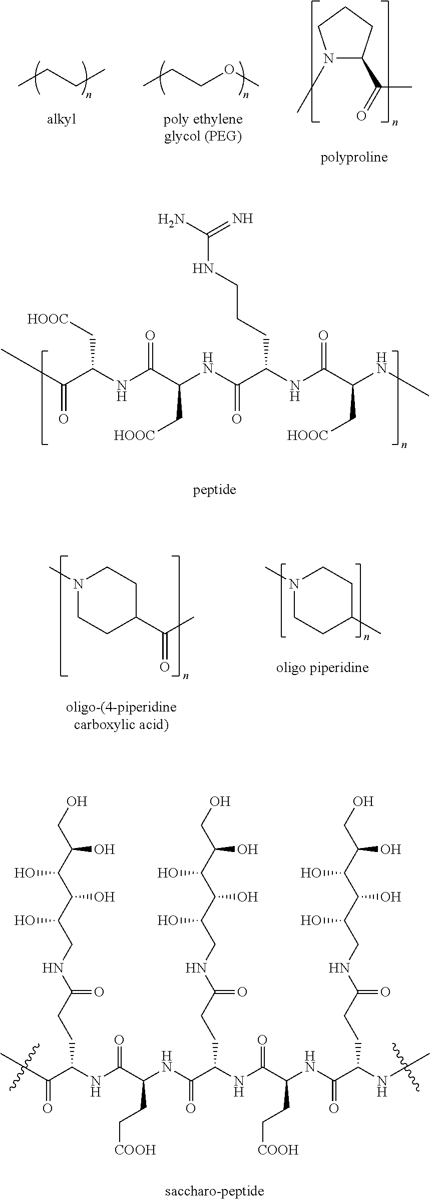

This disclosure further provides a method to rejuvenate an exhausted CAR T cell. The method comprises the steps of:
a. providing the exhausted CAR T cell a first component comprising a conjugate, wherein the conjugate comprises a targeting ligand covalently linked to a payload of drug through a releasable or non-releasable linker;
b. providing said exhausted CAR T cell a second component comprising a fusion receptor linked to the exhausted CAR construct, wherein the fusion receptor comprises a targeting ligand binding module and a membrane-anchoring module;
c. letting the targeting ligand binding module of the second component bind to the targeting ligand in the first component to form a complex,
d. letting the membrane-anchoring module mediate internalization of the complex into the exhausted CAR T cell;
e. letting the payload drug either block the inhibitory signaling of the exhausted CAR T, or re-activate said CAR T through an antigen independent pathway.

In some preferred embodiment, the aforementioned method carries a payload drug executing its function within the endosome of the exhausted CAR T, and the targeting ligand and the payload drug are linked by a nonreleasable linker.

In some preferred embodiment, the aforementioned method carries a payload drug executing its function as a free drug in the cytosol of the exhausted CAR T, and the targeting ligand and the payload drug are linked by a releasable linker.

In some preferred embodiment, the targeting ligand of the first component is folate, FITC or FK506 in aforementioned method.

In some preferred embodiment, the targeting ligand-binding module of the second component in aforementioned method is anti-FITC, folate receptor, or FKBP.

In some preferred embodiment, the targeting ligand-binding module of the second component in aforementioned method is Folate Receptor alpha (FRa).

In some preferred embodiment, the payload of drug of the first component in aforementioned method is a Toll Like Receptor 7 (TLR7) agonist or Simulator of interferon genes (STING) agonist.

In some preferred embodiment the payload of drug the first component in aforementioned method is an inhibitor to following proteins: SHP1/2, TC-PTP or DGKα, TGFβ.

In some preferred embodiment the Tur agonist in aforementioned method has the structure of

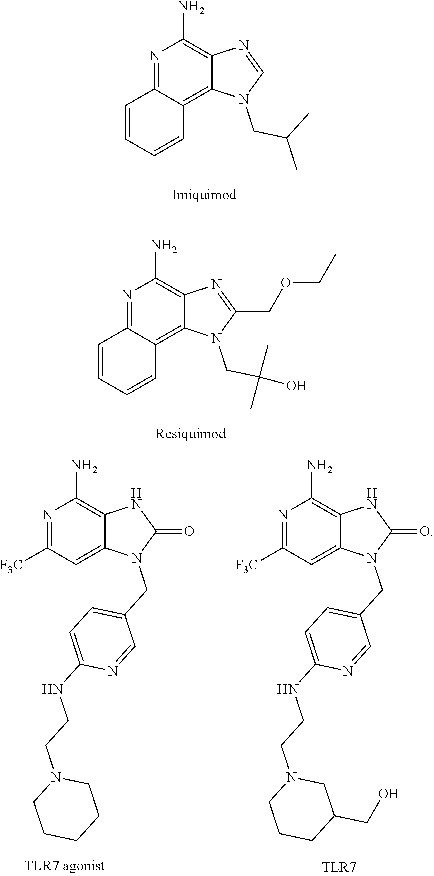 [SMILES: CC(C)Cn1cnc2c(N)nc3ccccc3c21 ; CCOCc1nc2c(N)nc3ccccc3c2n1CC(C)(C)O ; Nc1nc(C(F)(F)F)cc2c1[nH]c(=O)n2Cc1ccc(NCCN2CCCCC2)nc1 ; Nc1nc(C(F)(F)F)cc2c1[nH]c(=O)n2Cc1ccc(NCCN2CCCC(CO)C2)nc1]

In some preferred embodiment, the first component in aforementioned method is a Fluorescein-TLR7 agonist having the structure of

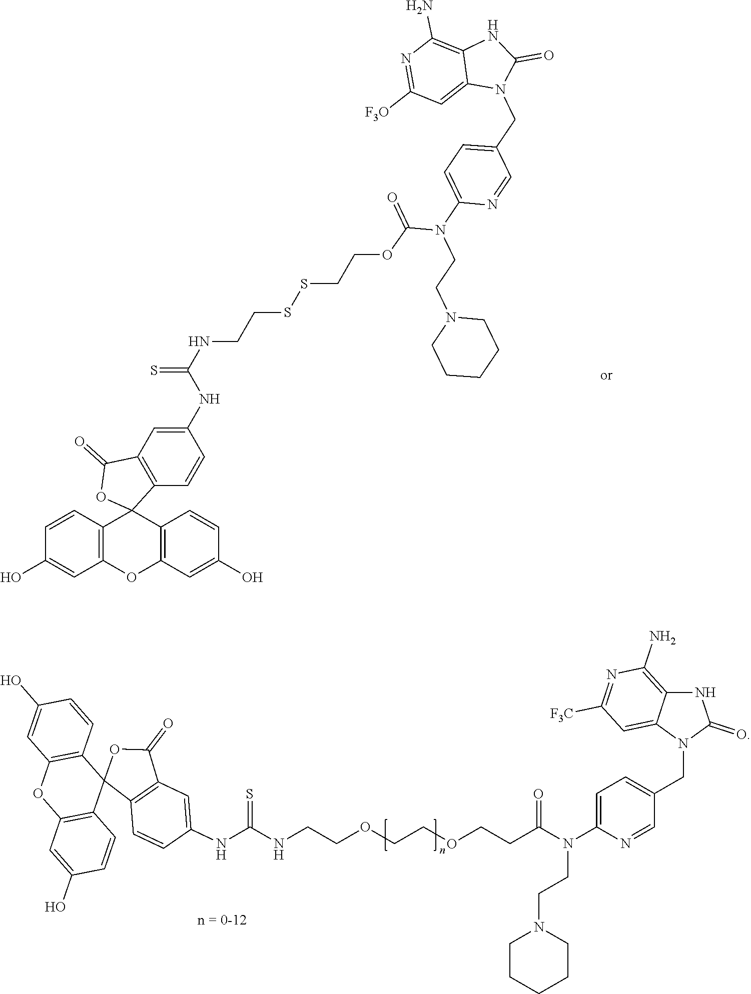

In some preferred embodiment, the first component in aforementioned method is a FK506-TLR7 agonist having the structure of

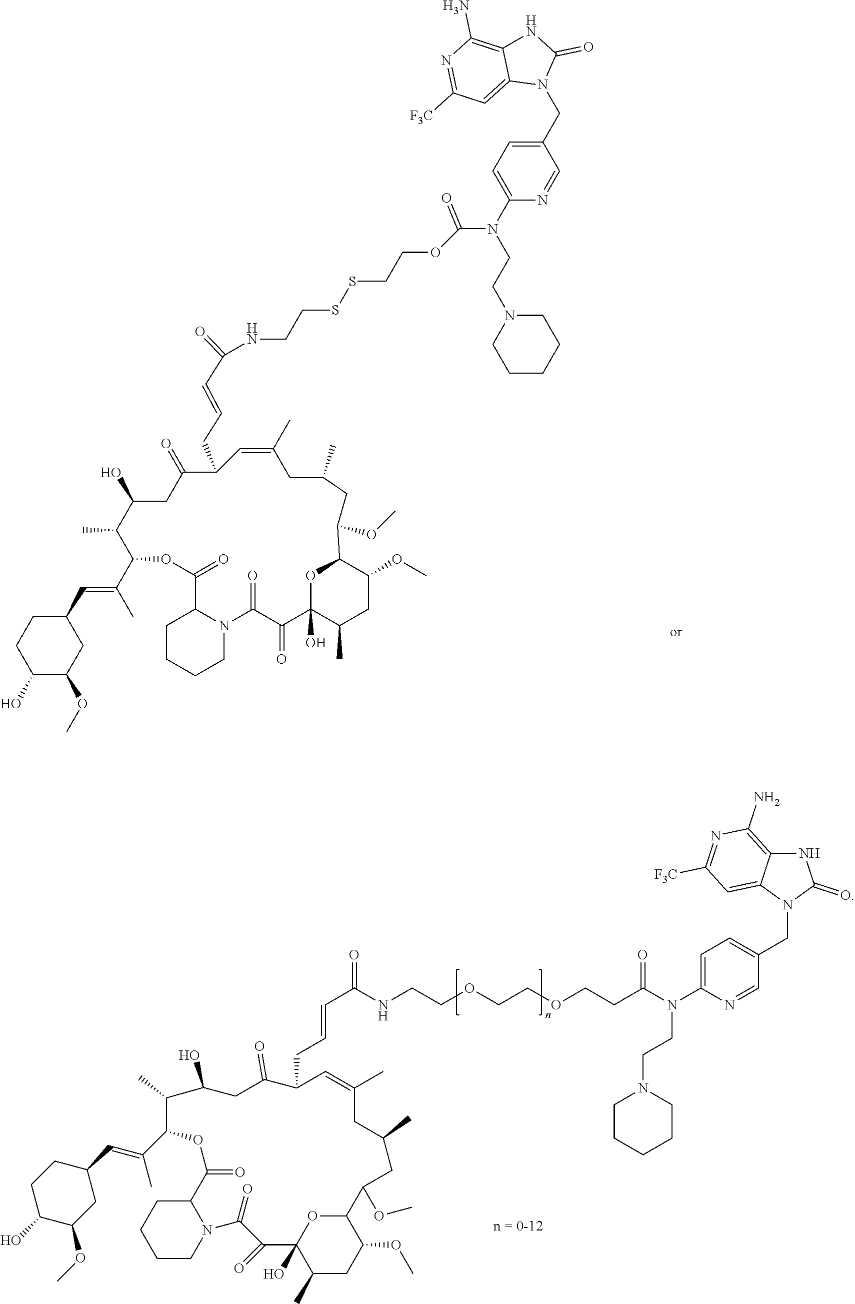

In some preferred embodiment, the first component in aforementioned method is

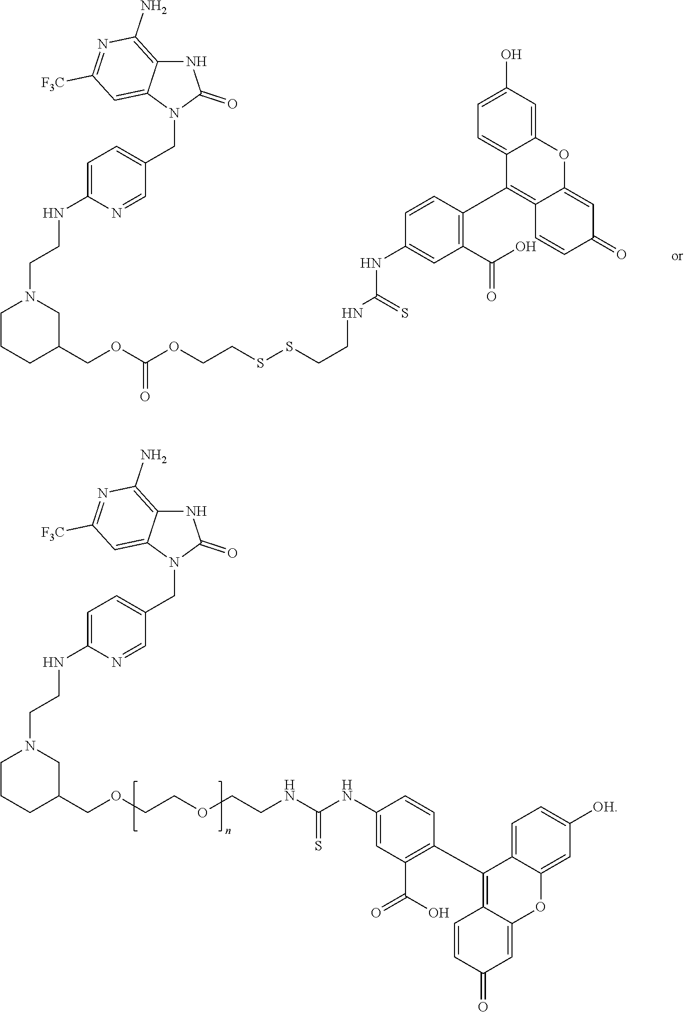

In some preferred embodiment, the first component in aforementioned method comprising payload drug selected from the group consisting of following TC-PTP phosphatase inhibitors:

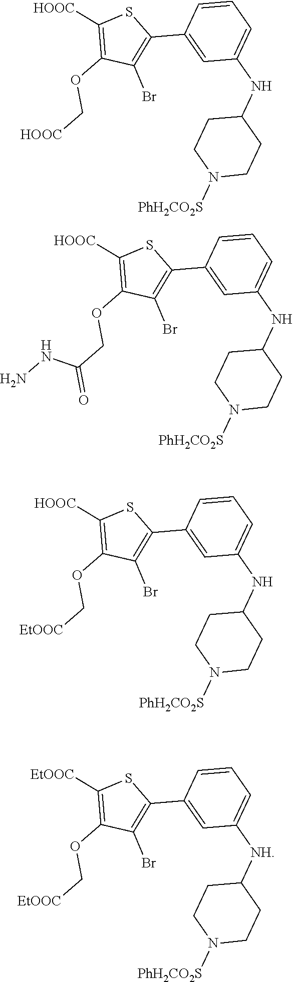

In some preferred embodiment, the first component in aforementioned method comprising the Phosphatase inhibitor connected to the fluorescein or FK506 (tacrolimus) to form the following structures:

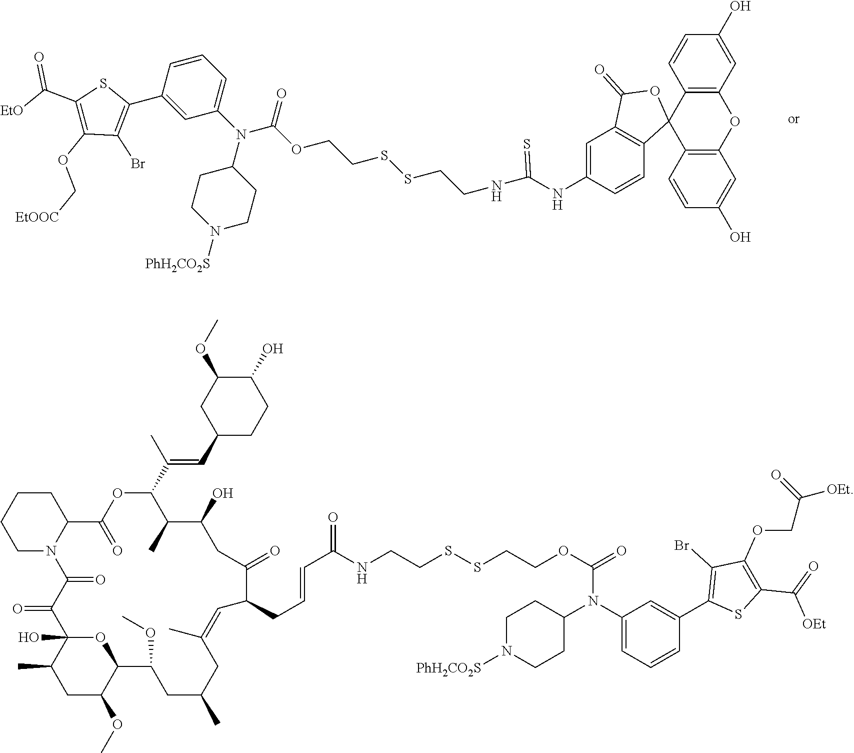

In some preferred embodiment, the first component in aforementioned method comprising a payload drug of a STING agonist of the following structures.

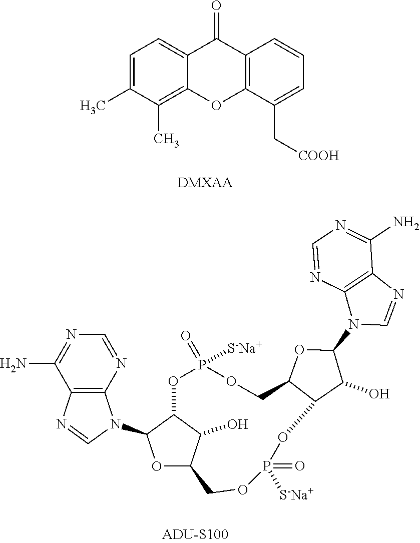

In some preferred embodiment, the first component in aforementioned method comprising a spacer between the targeting ligand and the payload drug that is selected from the group consisting of the following structures:

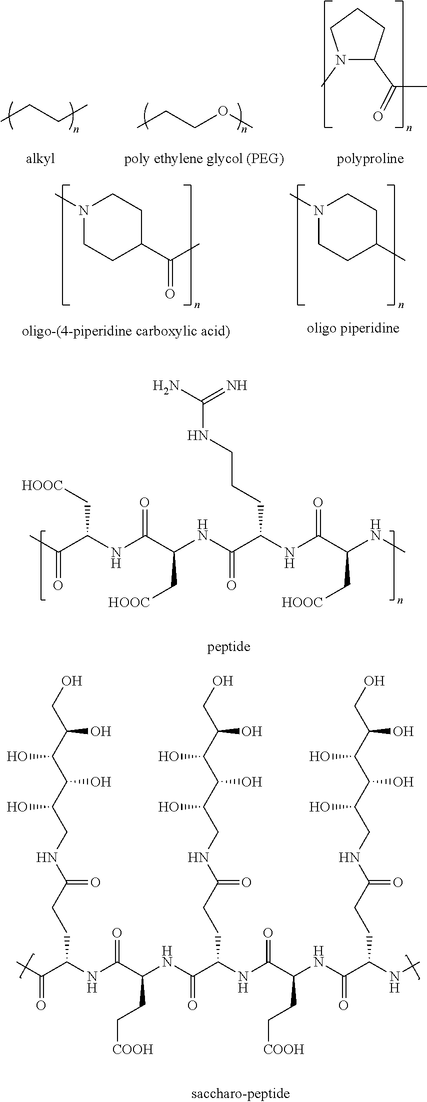

These and other features, aspects and advantages of the present invention will become better understood with reference to the following figures, associated descriptions and claims.

### BRIEF DESCRIPTION OF THE DRAWINGS

FIG. 1. 1a, Graph illustration of the exhaustion model. 1b, antiCD19 CART cell became exhausted after 3 times of stimulation of fresh Raji cell in vitro as shown by decreased killing effect.

FIG. 2. Graph illustration of the secret passageway. FKBP or antiFITC is linked to FRa as a fusion receptor, which constantly internalizes and delivers FK506 or FITC linked payloads into the cell.

FIG. 3. Binding affinity of FK506 and FITC to the corresponding fusion receptors. FK506-Rhodamine shows Kd=3.39 nM towards FKBP-FR fusion receptor, while FITC-AF647 shows Kd=8.03 nM towards antiFITC-FR.

FIG. 4. 4a. Structure of the TLR7 agonist. 4b. rejuvenation effect of TLR7 agonist on exhausted antiCD19 CART cell, shown as the increased killing effect, INFr level and decreased co-inhibitory molecule level.

FIG. 5. Chemical Structure of TLR7 agonists.

FIG. 6. in vitro model for the induction of CAR T cell exhaustion. (A) CD19⁺ Raji and anti-CD19 CAR T cells were co-cultured at 1:1 ratio with fresh Raji cells added every 12 h. (B) Lysis effect of CART cells gradually decreased as the number of stimulus (number of Raji cell addition) increases. CD19⁻K562 cells were used as control. (C) Expression level change of co-inhibitory molecules, PD-1, LAG3 and Tim3 for stim1 and stim3 in CD4 and CD8 positive CAR T cells. FIG. 7. Evaluation of TLR7 agonist and PTP1b inhibitor effect on rejuvenation of exhausted CAR T cells. (A) Chemical structure of TLR7 agonist and PTP1b inhibitor. (B-C) Exhausted CAR T cells were incubated with different concentrations of TLR7 agonist and PTP1b inhibitor monitored by lysis effect and INF_{γ} (B) and expression level of PD-1 LAG-3 and Tim3 after incubation (C). * denotes a p-value<0.05, **<0.01, ns=not significant.

FIG. 8. Evaluation of potential derivatization sites of the TLR7 agonist for non-releasable ligand targeted delivery. (A) Chemical structure of the TLR7 agonist analogs. (B) Exhausted CAR T cells were incubated with different concentration of TLR7 analogs and lysis effect was measured and compared to non-treated group.

FIG. 9. Design and evaluation of releasable and non-releasable targeted delivery of TLR7 agonist using HIV as a targeting ligand. (A) Illustration of chemical structure of FITC-TLR7 agonist. (B) Exhausted CAR T cells were incubated with different concentration of releasable FITC-TLR7 and non-releasable FITC-JTLR7 agonists and lysis effect was measured and compared to non-treated group. *denotes a p-value<0.05, **<0.01, ns=not significant. (C) Left, crystal structure of FITC (green) binding with FITC scFv (grey) (PDB: 1X9Q, left), the distance between FITC to the edge of FTIC scFv is measured to be around 18 Å. Right, similarly, crystal structure of R-848 (red) binding with TLR7 (grey) (PDB:5GMF) is shown with distance between R-848 and the edge of TLR7 around 24 Å. (D) Diagram illustrating the two possible working mechanisms, “Reaching” or “Jumping” Mode, for the non-releasable FITC-TLR7 agonists.

FIG. 10, Chemical linkers of variable rigidity and hydrophobicity available for usage in the design of targeting ligand-payload conjugates.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

Chimeric antigen receptor (CAR) T cell therapies have recently experienced substantial success in the treatment of several types of hematopoietic cancers. In the meantime, one should also recognize that some of the lymphoma and most solid tumor cases still have a very low response rate or a high relapse rate with CAR T cell therapies. This mainly result from one or combinations of the following three reasons: 1. Emergence of antigen negative cancer cell colonies under the selection pressure of CAR T cells, as seen in the case of CD19 negative ALL relapse treated with anti-CD19 CAR T cells; 2. Hindered initial homing, and proliferation of CART cells in solid tumor due to the aberrant tumor vasculature, dense stromal barrier and suppressive microenvironment; 3. Gradual exhaustion and lowered lysis effect of CAR T cells after continuous tumor antigen exposure. Assuming loss of antigen is not present for a given solid tumor patient (validated by biopsy sampling), the causes of a potential failure of a CAR T cell therapy are most likely to result from the latter two reasons. Therefore, to increase CAR T cells efficacy in solid tumors, practical methods for in vivo evaluation and rejuvenation of CAR T cells are highly desired.

Here we describe the novel design of a private passageway fusion receptor in CAR T cells as a universal platform to achieve both objectives: This FITC-FR fusion receptor is composed of two parts, scFv against FITC as the ligand binding domain at the N terminal and FRα as the GPI anchoring and internalizing domain at the C terminal. When independently expressed on CAR T cells, the FITC-FR fusion receptor can be specifically targeted by a FITC-immuno agonist to overcome the exhaustion status of CAR T cells in the suppressive tumor microenvironment. These immuno-agonists normally cause strong autoimmunity side effects, and can now be systemically dosed in a MC targeted form; and safely delivered to MC-FR positive CAR T cells. In the last few decades, great advances have been made in this field regarding the cell types, delivery methods and suitable diseases models: In terms of cell types, current cell therapies can be roughly categorized as chimeric antigen receptors (CARs), cell for tumor model and stem cell based regenerative medicine.

CAR T also known as chimeric T cell receptors, chimeric immunoreceptors or artificial T cell receptors, enable immune effector cells (usually T cells or NK cells) to recognize target cells with corresponding antigen and exercise their cytotoxic activity. The emergence and development of CAR-T technology provides promises to certain types of cancers which turns CAR-T into a superstar in the field of both biomedical research and clinical studies. Some traditional and improved CAR T cell design are disclosed in U.S. application Ser. No. 15/296,666, the content of which is incorporated herein entirely. In '666 application, a CAR system is produced by providing a cytotoxic lymphocytes expressing CARS that target a moiety that is not produced or expressed by cells of the subject being treated. This CAR system thus allows for focused targeting of the cytotoxic lymphocytes to target cells, such as cancer cells. The targeted moiety is part of a small conjugate molecule (SCM) that also comprises a ligand of a tumor cell receptor. Administration of a SCM along with the CAR-expressing cytotoxic lymphocytes results in the tailing of the cytotoxic lymphocyte response to only those cells expressing the tumor receptor to which the SCM is bound.

Despite the rapid progress of CAR T cell therapy in both research and clinical use field, there are concerns accompanied with CAR T therapy. One lethal side effect is cytokine storm generated from the fast lysis of tumor cells as well as it kills normal cells bearing CAR. In order to address such side effects, targeted delivery of CAR T cell with specific payload of drug to the target tumor cells to control such side effect is developed in PCT/US2018/018557, the content of which is incorporated herein entirely. Briefly, an engineered protein is coupled with a high affinity targeting ligand, wherein the targeting ligand carries at least one payload of drug to be internalized by the CAR T cell through the engineered protein to regulate transplanted cell therapy effects.

Another limitation of CAR T therapy is their tendency to get exhausted after repeated stimulation of cancer antigen. The reversibility of the exhausted phenotype of T cell is proven as T cells isolated from the solid tumor tissue show higher INF_{γ} secretion and killing effect if kept away from antigens (“rested”) overnight before re-stimulation⁴. However, it will be more appealing if rejuvenation can be achieved in a more clinical relevant way using drugs: either to block the inhibitory signaling or to activate the T cell through other pathways. Antibodies targeting checkpoint inhibitors (i.e. PD-1 CTLA-4 etc.) have shown some success in solid tumors in clinic, however, two or more targets in combination are often found to be necessary^{5 6}. Moreover, antibody therapy also suffers from poor penetration in solid tumor. Therefore, less reports have been seen for the combination therapy of CAR T and antibody for checkpoint inhibitors in solid tumor. Inhibiting the phosphatases, such as SHP1/2⁷ and TC-PTP⁸, that mediates TCR deactivating, is another way to block the inhibitory pathways. Both knockout experiments and small molecules inhibitors of these phosphatases have shown potent effect on lowering TCR threshold and increasing T cell activity, but none of them have been used in CAR T therapy. DGKα is another physiological inhibitor of TCR signaling and it's overexpressed in exhausted TIL. DGKα catabolize DAG to PA thereby reducing DAG levels, which results in attenuation of Ras and MARK ERK signaling. Inhibitor of DGKα recovers the degranulation and increases the killing effect of TIL and CAR T^{3 9}. Another approach to rejuvenate the T cell is to activate it through an antigen independent pathway. It has been known that certain pathogen pattern recognition (PPR) receptors, including Toll like receptors (TLR), do express on non-myeloid cell populations, including T cells, and can be activated in a similar way. Research has also shown that TLR2¹⁰, 4¹¹ and 7/8^{12 13} agonists can activate CD8 T cells and increase INF_{γ} secretion. However, due to the strong side effects of systemic dosing of TLR agonists¹⁴, none of these agonists have been used in CAR T therapy to re-activate the T cell or change the immunosuppressive microenviroment. It's also hindered by the controversial effect of TLR agonists on tumor cell itself¹⁵. Stimulator of interferon genes (STING) is a cytosolic DNA sensor (CDS) that widely expressed in hematopoietic cells in peripheral lymphoid tissues, including T cell, myeloid cells and monocytes. STING agonists have been used as an immune stimulator for many immunotherapies, and may also have a profound effect in CAR T therapy. However, although the mentioned inhibitors and agonists may have a profound rejuvenation effect on CAR T cell, ifs also highly possible that it may induce severe side effects if systematically dosed due to their highly potent pro-inflammatory functions. Therefore, a targeted delivery of the potential payloads to the CAR T cell is highly desired.

To solve the specific delivery problem, we designed a secret passageway platform, which can be expressed in T cell together with the CAR construct, so that certain payloads can be systemically dosed and specifically accumulated within the CAR T cell only, rendering other cells untouched. The system consists a fusion receptor and a classical CAR construct, linked through a T2A self-cleavable sequence. The fusion receptor contains two parts: (1) a ligand binding module, which can recognize a high affinity ligand-payload conjugate; (2) a membrane bound receptor module, which can mediate the internalization of the receptor/conjugate complex into the cell. Two protein/ligand pairs have been chosen for part 1, FKBP/FK506 and scFv against FITC (4M5.3)/FITC, for the following reasons: (1) the absence of FKBP or 4M5.3 on natural cell membrane guarantees the specific delivery of payload to fusion receptor positive CAR T cell, thus reduces the side effects to other cells; (2) the sub-nanomolar binding affinity between protein/ligand pairs promote sufficient payload accumulation inside the targeted cells. For the membrane bound receptor in part 2, Folate Receptor alpha (FRa) was chosen for its constitutive internalization properties regardless of Folate Acid binding¹⁶. We also designed a target-payload linkage system, where payload can be linked to targeting ligands with either non-releasable linker or a disulfide releasable linker, depending on the target. More specifically, since TLR7 locates in the endosome, secret passageway delivered TLR7 agonist can exert its function as soon as it enters the endosome through receptor mediated internalization. Therefore, releasable linker is not necessary in this case. While for other targets that are located in the cytosol instead of the endosome, such as SHP1/2, TC-PTP, DGK, TGFβ and STING, release of the free drug from the target-payload conjugate is necessary for its escape from the endosome. Together, this secret passageway system provides a versatile platform for specific in vivo delivery of numerous payloads to the exhausted CAR T cell.

These and other features, aspects and advantages of the present invention will become better understood with the following experiments examples.

### Methods

### Exhaustion of the AntiCD19 CAR T Cell and Drug Treatment:

AntiCD1.9 CAR T cell were co-cultured with Raji at 1:1 ratio in 6 well plate, while fresh Raji cell were added every 12 h to the same well. Killing effect and co-inhibitory markers were quantified by flow cytometry counting. For drug treatment, after 4 rounds of stimulation with Raji cells, exhausted antiCD19 CAR T were further incubated with drugs at different concentration for 12 h, and then quantified similarly.

### Targeting Ligand Binding Assay

Fusion receptor positive cells were incubated with certain ligand-dye molecule at different concentrations for 30 min at 4 degree. After incubation, cells were washed twice with PBS and then submitted to flow cytometry. MFI or percentage of shift is used for binding curve and calculation of Kd.

### Materials

### Cell Lines and Human T Cells

DMEM (Giber)) containing 10% heat-inactivated fetal bovine serum and 1% penicillin-streptomycin was used for the culture of MDAMB-231 and MDA-MB-231 CD19⁺ cells. Peripheral blood mononuclear cells (PBMCs) were isolated by Ficoll density gradient centrifugation (GE Healthcare Lifesciences, #17-5442-02) from human whole blood obtained from healthy volunteers. Pure CD3+ T cells were enriched from PBMCs using an EasySep™ Human T Cell Isolation Kit (STEM CELL technologies, #17951).

### Evaluation of Potential Payloads for In Vitro Rejuvenation of Exhausted CAR T Cells

anti-CD19 CAR T cells were co-incubated with CD19⁺ Raji cell at 1:1 ratio in 12-well plate at density of 2×10⁶ CAR T and 2×10⁶ Raji per well, new Raji cell were added every 12 h for 3 times, Raji cell population, lysis effect and co-inhibitory receptors were then tested to confirm the exhaustion of the CART cells. Both flow cytometry and luciferase-based assays were used to quantify the lysis effect. To test the rejuvenating efficacy of the potential payloads, this cell mixture was then transferred to 96-well plate, around 2×10⁵ cells per well, and different concentration of drugs were added. After 12 h, Raji cell population, lysis effect and co-inhibitory receptors were tested again and compared to the PBS treatment group.

### EXAMPLES

### Example 1. Exhaustion of the AntiCD19 CAR T Cell In Vitro

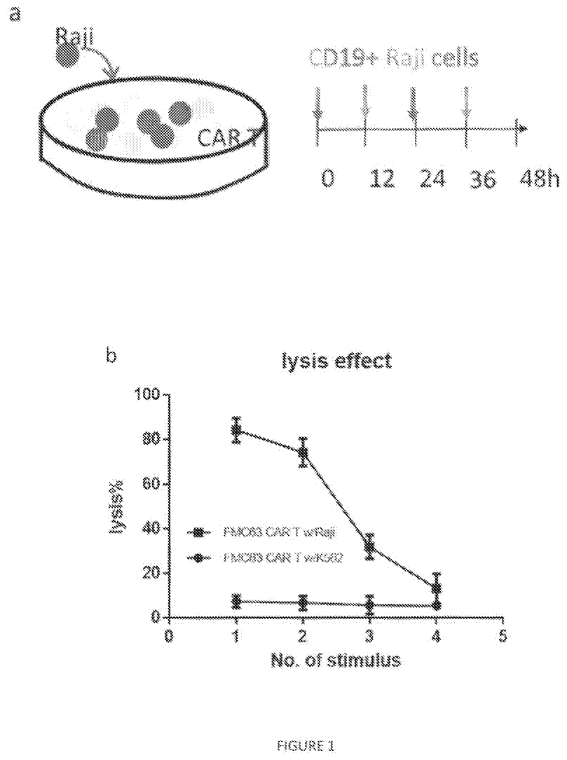

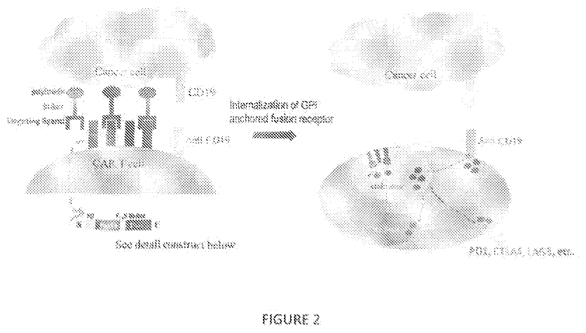

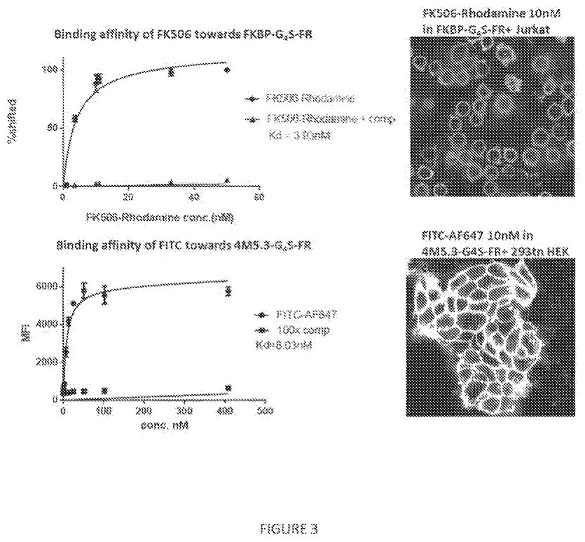

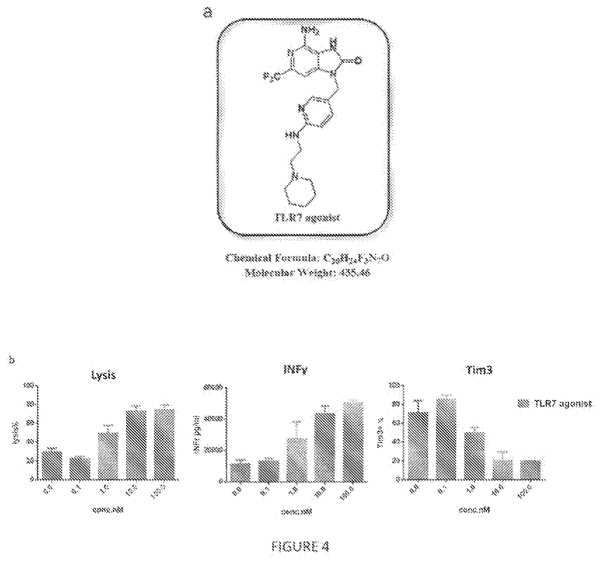

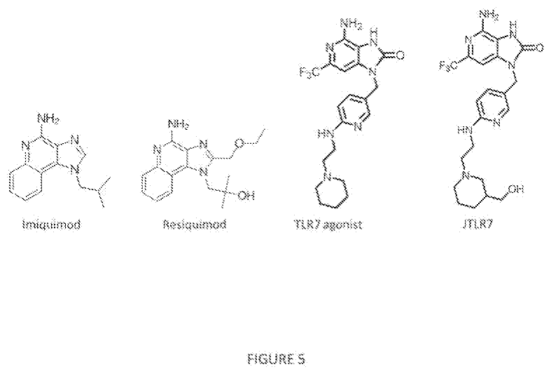

In this Example, we illustrated a model of exhausted CAR T cell. Briefly, FIG. 1a shows Raji cells, a type of B-lymphoma cells were co-cultured with FMC63 CAR T cells, a type of anti CD19 CAR T cells as described in the method section, and fresh Raji cells were added to the co-culture every 12 hours for consecutive three days. FIG. 1b shows these CAR T cells became exhausted after 3 times of stimulation by fresh Raji cells in vitro, indicated by decreased killing effect. Co cultured K562 cells served as negative control.

### Example 2. Design of the Secret Passageway

In this Example, we show a graphic illustration of the secret passageway of delivery payload drug to targeted cell types. FKBP or antiFITC is linked to FRa as a fusion receptor, which is able to engage a targeting ligand FK506 or FITC linked to a specific payload of drug. Due to the nature of FRa, it constantly internalizes and delivers FK506 or FITC linked payloads into the CAR T cell. Therefore, when CAR T is engaged to its target cells through CAR, in this example, anti-CD 19 molecule on the CAR T surface engages CD-19 of the cancer cell, the delivered payload drug inside of CAR T may execute its function, i.e. regulate CAR T activity based on the payload function. For example, some payload drugs may be directed to act on PD-1, CTLA4, or LAG3 T cell function regulatory molecules to rejuvenate CAR T when necessary.

### Example 3. Reversion of the Exhausted AntiCD19 CAR T Cell by TLR7 Agonist

In this Example, we show that targeting ligand FK506 or FITC successfully engages its respective fusion receptor (FKBP or FITC-AF647 linked to Folate Receptor by G₄S). In this example the payload drug is an imaging agent Rhodamine to show the payload distribution in fusion receptor transfected Jurkat cells. The binding affinity FK506 and FITC are calculated by competitive binding and FK506-Rhodamine shows Kd=3.39 nM towards FKBP-FR fusion receptor, while FITC-AF647 shows Kd=8.03 nM towards antiFITC-FR

### Example 4. Design of Potential Releasable and Non-Releasable FK506-TLR7 Agonist and FITC-TLR7 Agonist

In this Example, we provided targeting ligand FK506 conjugated to a Toll Like Receptor 7 agonist with the structure below to treat exhausted CAR T cells. A dose dependent pattern of increased killing effect is observed when TLR7 agonist payload drug is targeted to exhausted CAR T cells. Accordingly, the indicator of CAR T activity, IFN_{γ} expression level, increased in a dose dependent manner relative to payload drug concentration; and the expression level of a T cell effect inhibitory molecule, Tim 3, had showed a reverse dose dependence manner, i.e. Tim 3 expression decreased as the concentration of payload drug is increased.

FK506-TLR7 agonist conjugate should have one of the structures given below:

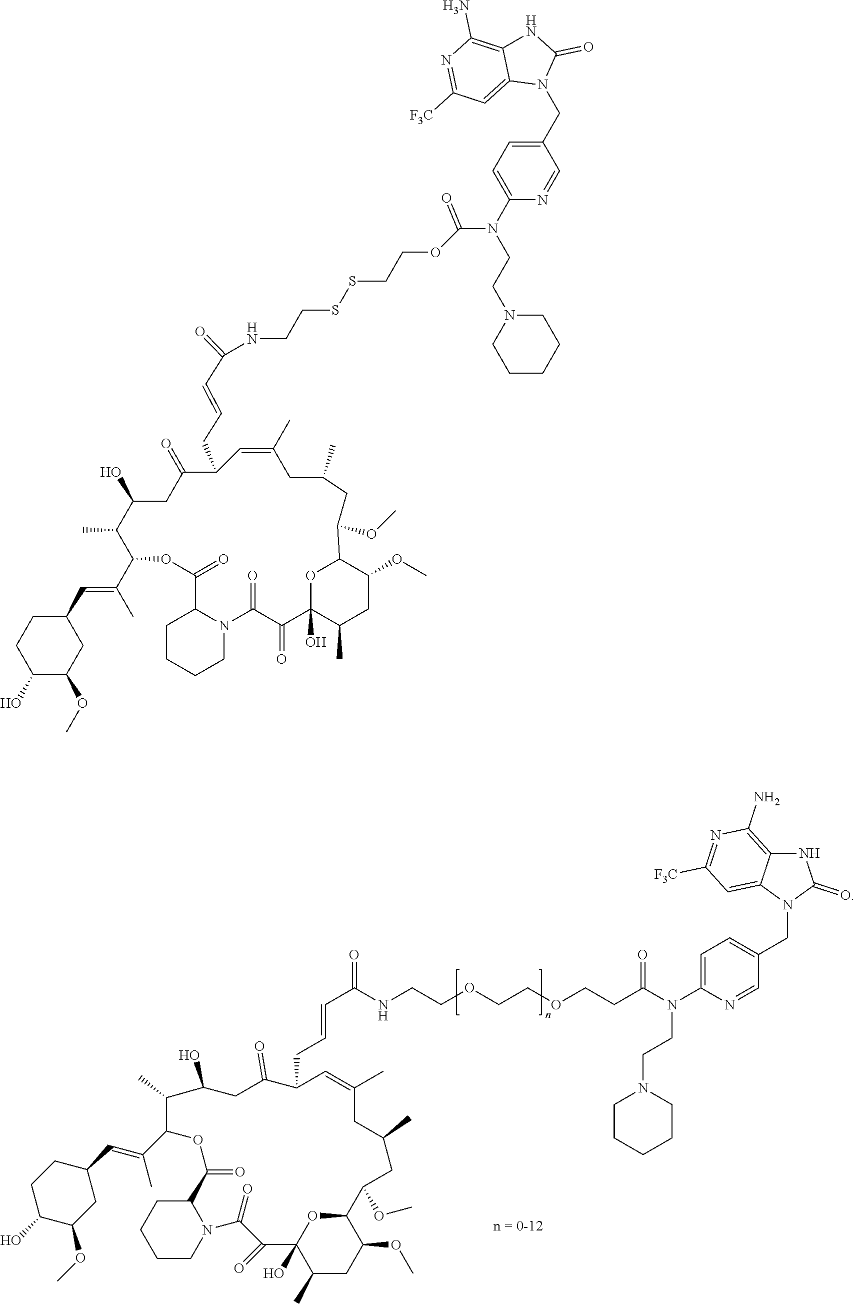

Similarly, Fluorescein-TLR7 agonist conjugate should have one of the structures given below and rejuvenate exhausted CAR T if the functional CAR T has an anti-FITC fusion receptor.

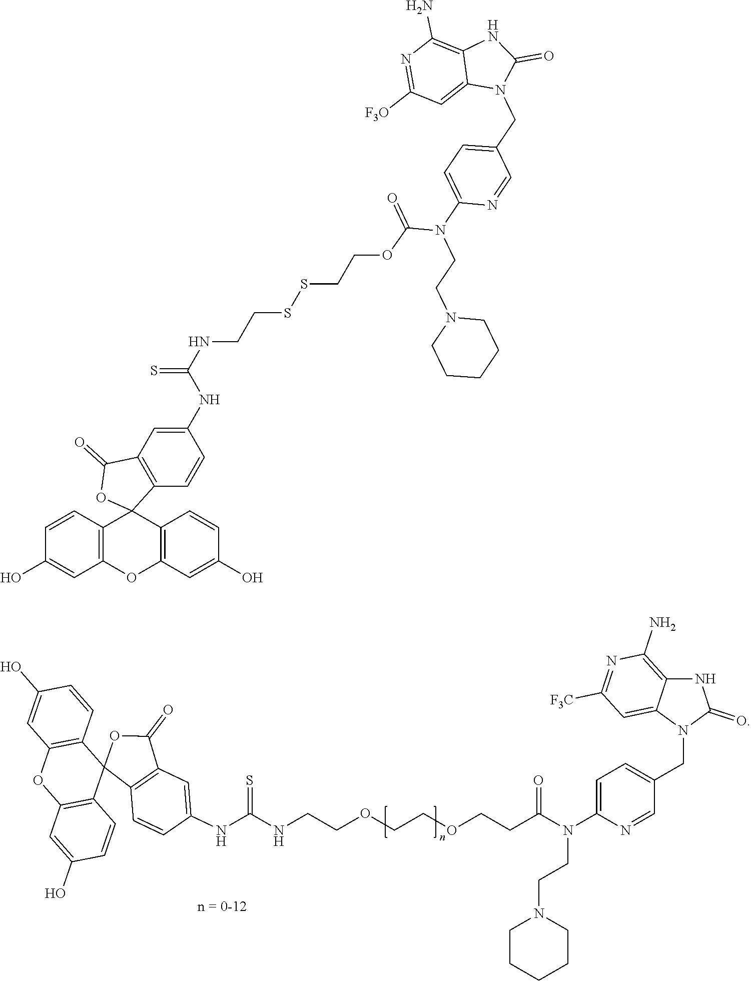

### Example 5. Design of Other Potential Payloads for the Rejuvenation of the Exhausted CAR T Cell

In this Example we provide a list of structures of other potential payloads that may revert CAR T exhaustion with lower nanomolar range potency.

1) TC-PTP phosphatase inhibitor should have the following structures,

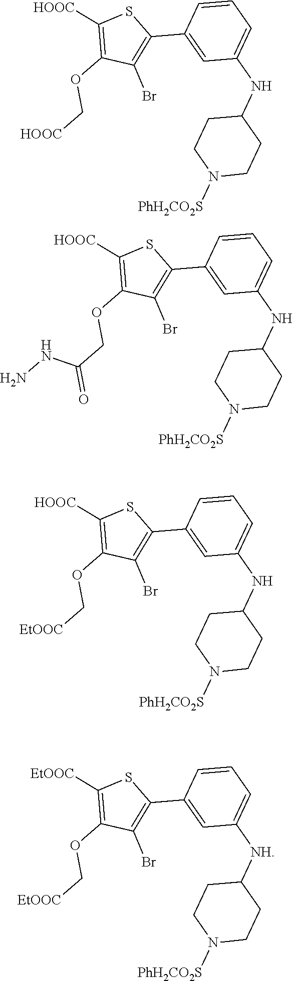

Phosphatase inhibitor mentioned above may be connected to the fluorescein or FK506 (tacrolimus) in the following way.

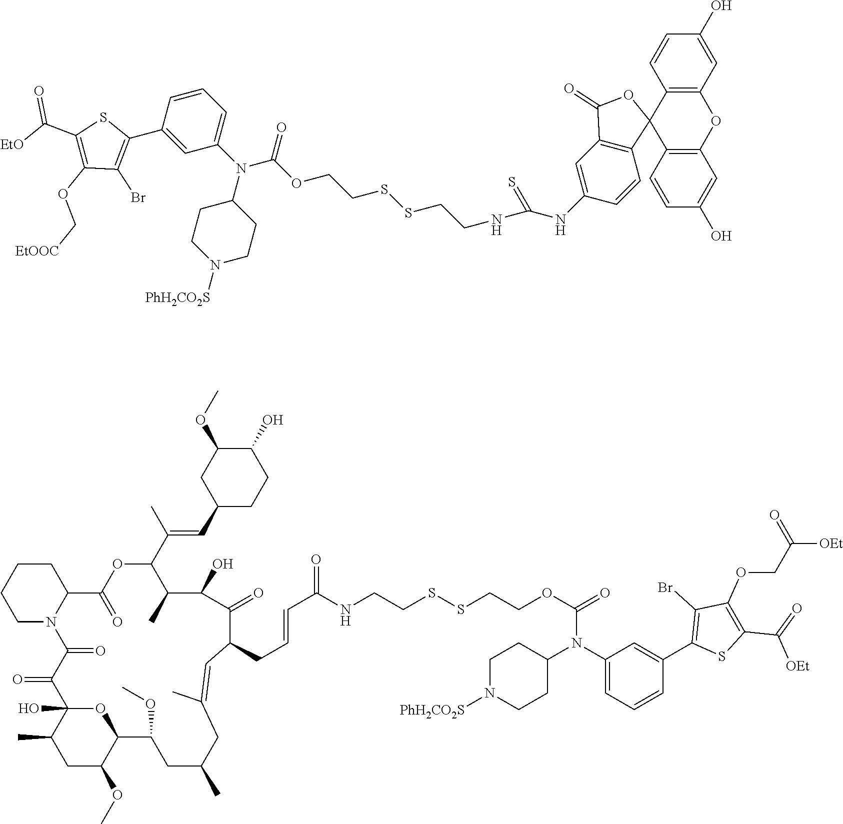

2) The STING agonist should have the following structure,

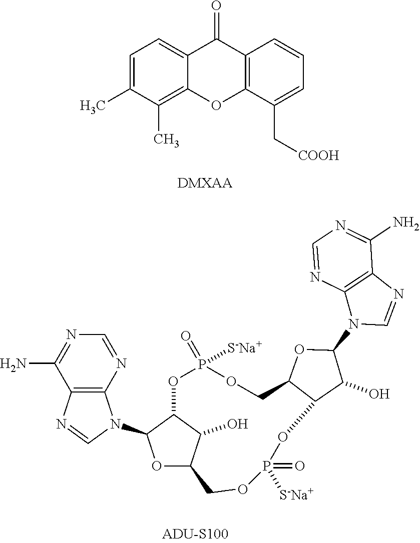

### Example 6. Design of Spacers Between Targeting Ligands and the Potential Payloads

Below are a list of spacers that can be employed to link the targeting ligands and any potential payloads.

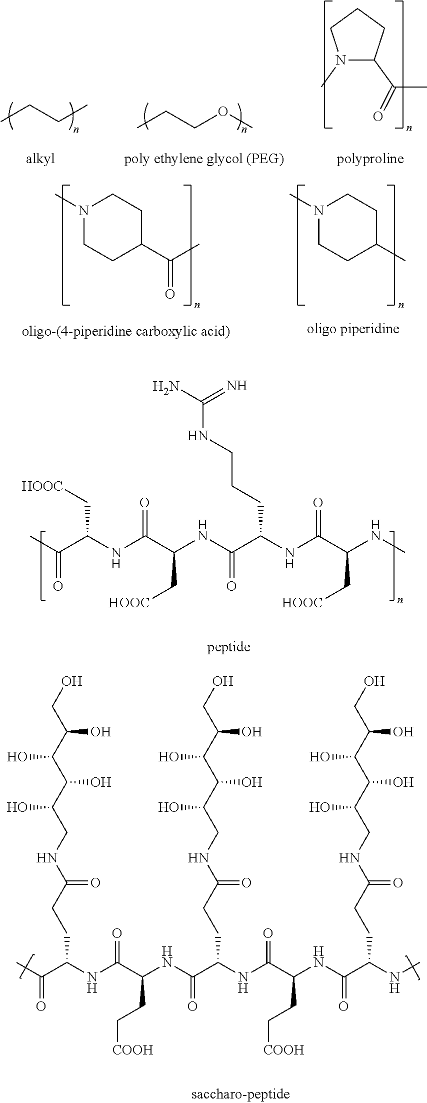

### Example 7. Evaluation of the Ability of Phosphatase Inhibitors and TLR7 Agonists to Rejuvenate Exhausted CAR T Cells

One major limitation of CAR T cell therapies in solid tumors is their tendency to become exhausted after repeated stimulation with cancer antigens. This phenomenon however, is not specific to CAR T cells, but has been described in both chronic virus infections⁴ and tumor infiltration lymphocytes⁵. The reversibility of the exhausted phenotype of T cells has been proven in studies where T cells isolated from the solid tumor tissue show a higher INF_{γ} secretion and a killing effect if kept away from antigens (“rested”) overnight before re-stimulation⁵⁷. However, it would will be more appealing if rejuvenation could be achieved in a more clinically relevant way using commercially available therapeutics: either to block the inhibitory signaling or to activate the T cells through other pathways. Antibodies targeting checkpoint inhibitors (i.e. PD-1, CTLA-4, etc.) have shown some success in solid tumors in-clinic¹⁵¹, however, two or more targets in combination often have been found to be necessary. Moreover, antibody therapy also suffers from poor penetration in solid tumors and this may have led to less reports for the combination therapy of CAR T cells and checkpoint blockades (ICB) in solid tumors.

The inhibition of the phosphatases, such as SHP1/2 and TC-PTP, that mediate TCR deactivation, is a potential way to block tonic CAR T signaling. SHP1/2 phosphatase is responsible for mediating the signal from PD-1 and other exhaustion markers. Data has shown that SHP1/2 phosphatase inhibitor or silencing can increase the activity of T cells and CAR T cells⁶⁻⁹. TC-PTP is known to be an important player in T cell activity signaling. Mice harboring a T cell specific TC-PTP deficiency have increased susceptibility to inflammation and autoimmunity due to heightened antigen-driven T cell activation. TC-PTP inactivates Src family kinase downstream of the TCR, thereby contributing to the threshold of TCR activation¹¹. Although both knockout experiments and small molecule inhibitors of these phosphatases have shown potent effect on lowering TCR threshold and increasing T cell activity, none of them have been used in CAR T therapy. A representative SHP1/2 inhibitor has the structure of

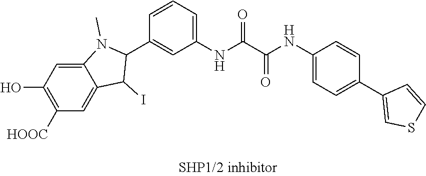 [SMILES: CN1c2cc(O)c(C(=O)O)cc2C(I)C1c1cccc(NC(=O)C(=O)Nc2ccc(-c3ccsc3)cc2)c1]

Another approach to rejuvenate the T cells is to augment their activity through the engagement of antigen independent innate immune receptors. It has been known that certain pathogen pattern recognition (PPR) receptors, including toll like receptors (TLR), do express on non-myeloid cell populations, including T cells, and can be activated in a similar way. Research has also shown that co-stimulation of TLR7/8 agonists and TCR signaling can activate CD8 T cells and increase INF_{γ} secretion¹³. However, due to the strong side effects of systemic dosing of TLR agonists, none of these agonists have been used in CAR T therapy to reactivate the T cell or change the immunosuppressive microenvironment. The employment of TUR agonists for cancer immunotherapy is also hindered by the controversial effect of TLR agonists on the tumor cells. Therefore, a targeted delivery of the potential payloads to the CAR T cell is highly desired. A potent TLR7 agonist was found in the literature (FIG. 7A) which is around 40 fold stronger than the FDA approved imiquimod.

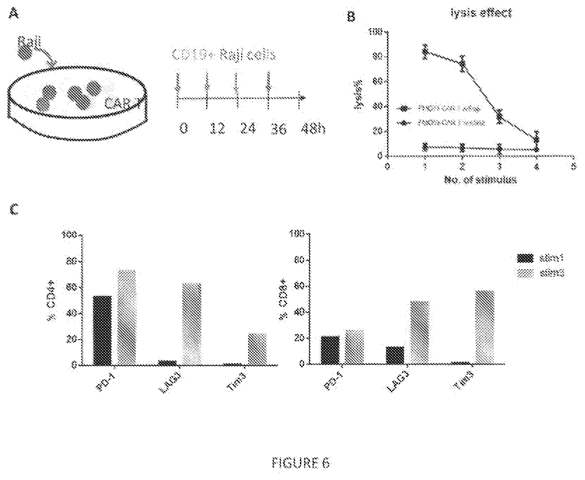

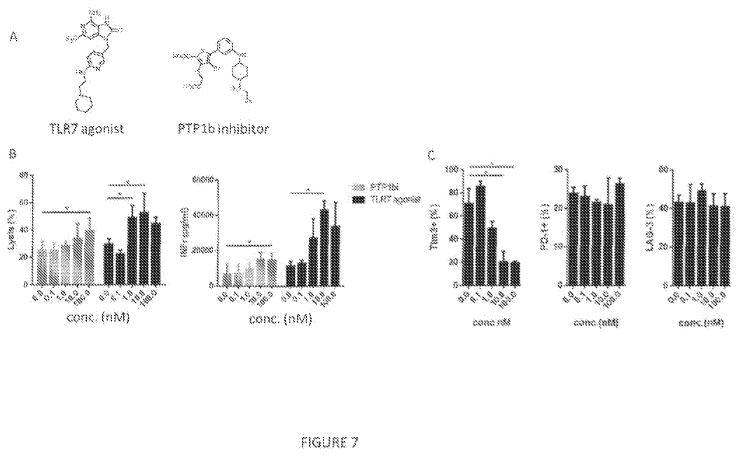

To set up an in vitro screening model, as shown in FIG. 6, anti-CD 19 CAR T cells were exposed to 4 rounds of addition of CD19 positive Raji cells, and became exhausted as marked by gradual decreased lysis activity as well as increased co-inhibitory markers in an in vitro co-culture model. It's worth noticing that the culture medium is important for the introduction of CAR T exhaustion and needs to be kept the same without new replenish or change during the whole process. It indicates that the soluble components that are released by cancer cells and/or CAR T cells into the medium, most likely immunosuppressive cytokines and modulators (adenosine etc.), play a pivotal role in this process. It also suggests that the exhaustion of CAR T cells generated by this in vitro model is at a rather pliable than irreversible status.

Treatment of the TLR7 agonist and PTP1b (highly homologous to TC-PTP²⁶) inhibitor²⁷ of choice with the already exhausted CAR T cells was shown to be able to reactivate them compared to the no treatment group (FIG. 7). No significant changes, however, were observed in the expression level of co-inhibitory markers except for Tim3. As shown by FIG. 7C, the PTP1b inhibitor in general does not show as strong of a reactivation effect as the TLR7 agonist. Without being limited by any theory, this result could be due to the current in vitro screening model where “Reversion” rather than “Prevention” of exhaustion is studied, and the phosphatases are already “silenced” at the exhausted status, therefore their inhibition will have little to no effect. A modified screening model for a future study will test the effects of phosphatase inhibitors and other drugs with a focus on the “prevention” of exhaustion by adding the drugs at the beginning of all cultures and keeping the rest of the settings the same. In this way, it may be possible to see whether the inhibition of phosphatase can lower the tonic signaling of CAR T while still keeping a functional killing effect. We will mainly focus on TLR7 agonist for the following study.

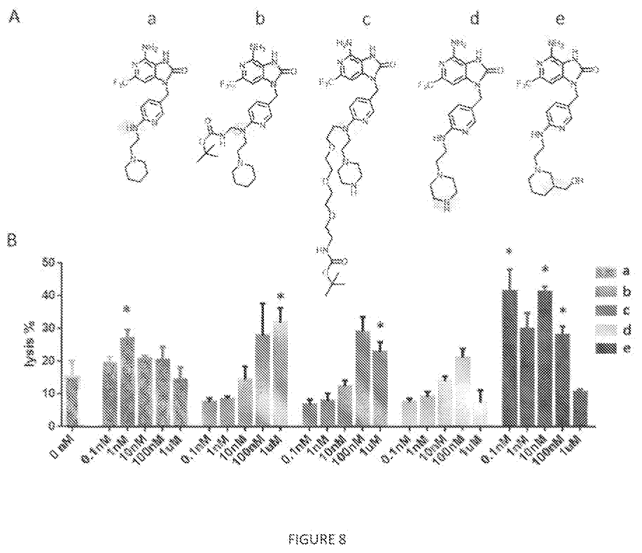

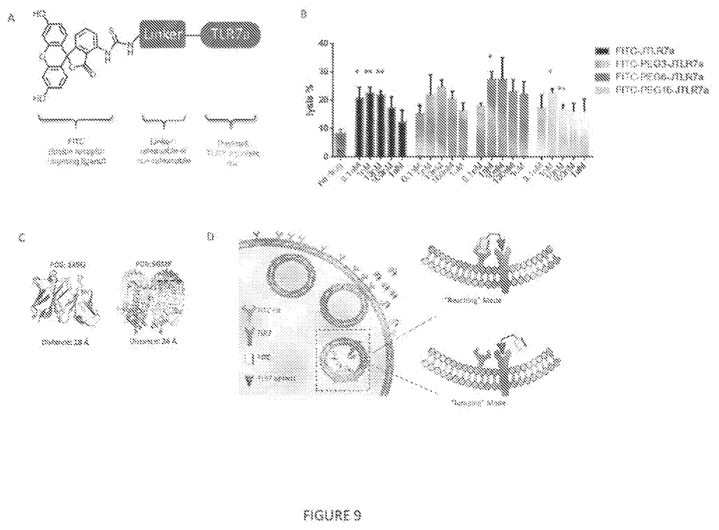

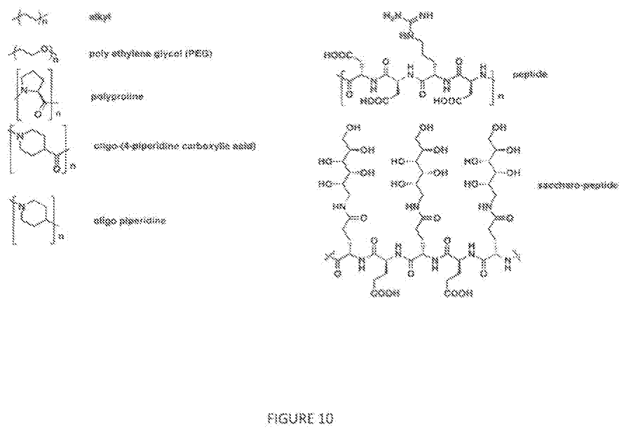

Since TLR7 is one of the 4 TLR family members that resides inside the endosome, it is speculated that a non-releasable linker between the TLR7 agonist and our secret passageway targeting ligand would preserve its TLR7 agonist function²⁸. To achieve that, several TLR7 agonist analogs were prepared and tested to find the proper derivatization sites for linkage. As shown in FIG. 8, the TLR7 agonist with a CH₂OH extension at the piperidine ring has an even higher activity compared to the parent drug. Therefore, this derivative site will be used for a non-releasable conjugate. A disulfide bond linked self-immolative form also has been synthesized. In order to understand the distance needed for this TLR7 agonist to reach its own target, three different lengths of linker (PEG3, 6, 16) between the FITC and TLR7 agonist were made for the non-releasable FITC-TLR7. As shown in FIG. 9 C, all of the non-releasable forms had some effect, while the PEG₆ compound showed the best dose-dependent response. These results indicate that TLR7 agonists may dock with TLR7 either by reaching out while binding with FITC-FR or jumping between TLR and FITC-FR, under which conditions the length of the linker in between is not a crucial factor (FIG. 9 D-E). Since the non-releasable FITC-TLR7 is trapped inside the endosome and the volume of each endosome is much smaller than the cytosol, the intra-endosome TLR7 can get to its functional concentration much faster and quicker, resulting in a smaller IC₅₀.

### Example 8. Other Potential Payloads for Revert/Prevent the Exhaustion of CAR T Cells

Other than TLR7 agonist, there are several other potential payloads that may revert/prevent the exhaustion of CAR I cells as described below. Some of the targets may not have agonists or inhibitors with IC₅₀ suitable for our targeted drug delivery approach for now, but are still worth noticing and may be explored through other inhibitory mechanisms, such as CRISPR or targeted microRNA delivery approaches.
STING agonist
   The Simulator of IFN Genes (STING) is a master adaptor involved in cytosolic DNA sensing and the following IFN-β production. STING associates weakly to sdDNA, but strongly binds the endogenous cyclic dinucleotide GMP-AMP (cGAMP) synthesized by the cGMP-AMP synthase (sGAS). It is predominantly expressed in macrophages, T cells, a variety of DCs, endothelial cells, and select fibroblasts and epithelial cells. Studies of STING have mainly focused on its function in macrophages and dendritic cells, and recently some groups have noticed the direct effect of STING activation in T cells⁴⁰. It is possible that a STING agonist will have a similar pro-inflammatory effect on T cells. ADU-S100 is one of the many STING agonists that has been pursued in clinics.
DGK-α inhibitor
   Diacylglycerol Kinase-α (DGK-α) converts diacylglycerol (DAG), a second messenger in TCR signaling together with IP3, to phosphatidic acid (PA). DGK is more highly expressed in CD8TIL than in CD8-NIL, and its inhibition promotes ERK phosphorylation and lytic degranulation⁴¹; it also restores lytic functions of CAR TIL that are isolated from in vivo⁵. Some DGK inhibitor structures are as following;

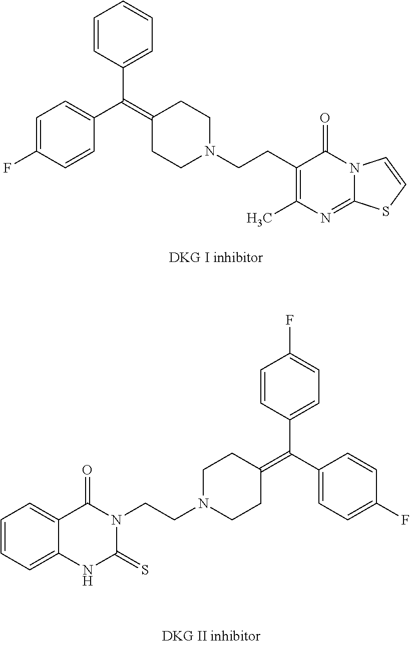 [SMILES: Cc1nc2sccn2c(=O)c1CCN1CCC(=C(c2ccccc2)c2ccc(F)cc2)CC1 ; O=c1c2ccccc2[nH]c(=S)n1CCN1CCC(=C(c2ccc(F)cc2)c2ccc(F)cc2)CC1]

TGFβRI (ALK5) inhibitor
   TGFβ is known for its immunosuppressive function in many immune cells, such as the T cell, B cell, and macrophages. The blockage of TGFβ type I receptor (TGFβRI, also called ALK5) in T cells reverts the immunosuppressive environment of the tumor⁴². Small molecule inhibitors have been pursued with galunisertib (LY2157299 monohydrate) and EW-7197 tested in clinics⁴³⁻⁴⁴.

A TGFβ inhibitor structure is as following:

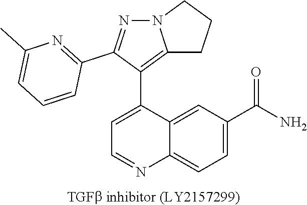 [SMILES: Cc1cccc(-c2nn3c(c2-c2ccnc4ccc(C(N)=O)cc24)CCC3)n1]

EZH2 inhibitor
   Enhancer of Zeste Homolog 2 (EZH2) is a histone H3K27 methyltransferase with a strong correv the Treg function. Genetic or pharmacological disruption of EZH2 drove acquisition of proinflammatory function of tumor infiltrating Treg⁴⁵. Since exhausted CTL in chronic virus infections is also characterized by unique epigenetic changes¹⁷⁴, it is possible that EZH2 inhibitors will be able to reverse this exhaustion status. Several small molecules of EZH2 inhibitors have been developed, including CPI1205, EPZ6438 and GSK126.

Therefore, the FITC-FR fusion receptor and the corresponding FITC targeted immune-agonists payloads provide a universal platform for the monitor and control of CAR T cells homing and persistence in solid tumor. This approach can be easily incorporated into CAR T cells for any antigen since the FITC-FR fusion receptor is independently expressed to the CAR construct. The modular design of targeting ligand-payload conjugates also makes it easier for the switching and modification. This approach combines the benefits of cell therapy and small molecule-based targeted drug delivery and may require extra characterization of both the engineered cells and the corresponding ligands.

The success of CAR T cells in solid tumor most likely requires the combination of multiple approaches targeting other players within the microenvironment as well, such as breaking down of the extracellular matrix by PI3K kinase inhibitors, reprograming of anti-inflammatory M2 macrophages to a proinflammatory M1 phenotype, and upregulation of the decreased MHC molecules level on cancer cells. Therefore, more and more combinational therapy studies will be conducted both preclinically and clinically. However, at the same time, a careful examination and control of the CAR T cell itself cannot be neglected and should be optimized by using simple but robust systems like FITC-FR fusion receptors in preclinical research first before it reaches to humans.

### REFERENCES

1. Park, J. H.; Riviere, I.; Gonen, M.; Wang, X.; Senechal, B.; Curran, K. J.; Sauter, C.; Wang, Y.; Santomasso, B.; Mead, E.; Roshal, M.; Maslak, P.; Davila, M.; Brentjens, R. J.; Sadelain, M., Long-Term Follow-up of CD19 CAR Therapy in Acute Lymphoblastic Leukemia. The New England journal of medicine 2018, 378 (5), 449-459.
2. Jiang, Y.; Li, Y.; Zhu, B., T-cell exhaustion in the tumor microenvironment. Cell Death Dis 2015, 6, e1792.
3. Matsuzaki, J.; Gnjatic, S.; Mhawech-Fauceglia, P.; Beck, A.; Miller, A.; Tsuji, T.; Eppolito, C.; Qian, F.; Lele, S.; Shrikant, P.; Old, L. J.; Odunsi, K., Tumor-infiltrating NY-ESO-1-specific CD8+ T cells are negatively regulated by LAG-3 and PD-1 in human ovarian cancer. Proceedings of the National Academy of Sciences of the United States of America 2010. 107 (17), 7875-80.
4. Moon, E. K.; Ranganathan, K.; Eruslanov, E.; Kim, S.; Newick, K.; O'Brien, S.; Lo, A.; Liu, X.; Zhao, Y.; Albelda, S. M., Blockade of Programmed Death 1 Augments the Ability of Human T Cells Engineered to Target NY-ESO-1 to Control Tumor Growth after Adoptive Transfer. Clinical cancer research: an official journal of the American Association for Cancer Research 2016, 22 (2), 436-47.
5. Moon, E. K.; Wang, L. C.; Dolfi, D. V.; Wilson, C. B.; Ranganathan, K.; Sun, J.; Kapoor, V.; Scholler, J.; Pure, E.; Milone, M. C.; June, C. H.; Riley, J. L.; Wherry, E. J,; Albelda, S. M., Multifactorial T-cell hypofunction that is reversible can limit the efficacy of chimeric antigen receptor-transduced human T cells in solid tumors. Clinical cancer research: an official journal of the American Association for Cancer Research 2014, 20 (16), 4262-73.
6. Chae, Y. K.; Arya, A.; Iams, W.; Cruz, M. R.; Chandra, S.; Choi. J.; Giles, F., Current landscape and future of dual anti-CTLA4 and PD-1/PD-L1 blockade immunotherapy in cancer; lessons learned from clinical trials with melanoma and non-small cell lung cancer (NSCLC). J Immunother Cancer 2018, 6 (1), 39.
7. Ott, P. A.; Hodi, F. S.; Kaufman, H. L.; Wigginton, J. M.; Wolchok, J. D., Combination immunotherapy: a road map. J Immunother Cancer 2017, 5, 16.
8. Watson, H. A.; Dolton, G.; Ohme, J.; Ladell, K.; Vigar, M.; Wehenkel, S.; Hindley, J.; Mohammed, R. N.; Miners, K.; Luckwell, R. A.; Price, D. A.; Matthews, R. J.; Ager, A., Purity of transferred CD8(+) T cells is crucial for safety and efficacy of combinatorial tumor immunotherapy in the absence of SHP-1. Immunology and cell biology 2016, 94 (8), 802-8.
9. Wiede, F.; Shields, B. J.; Chew, S. H.; Kyparissoudis, K.; van Vliet, C.; Galic, S.; Tremblay, M. L.; Russell, S. M.; Godfrey, D. I.; Tiganis, T., T cell protein tyrosine phosphatase attenuates T cell signaling to maintain tolerance in mice. The Journal of clinical investigation 2011, 121 (12), 4758-74.
10. Prinz, P. U.; Mendler, A. N.; Masouris, I.; Durner, L.; Oberneder, R.; Noessner, E., High DGK-alpha and disabled MAPK pathways cause dysfunction of human tumor-infiltrating CD8+ T cells that is reversible by pharmacologic intervention. Journal of immunology 2012, 188 (12), 5990-6000.
11. Chua, B. Y.; Olson, M. R.; Bedoui, S.; Sekiya, T.; Wong, C. Y.; Turner, S. J.; Jackson, D. C., The use of a TLR2 agonist-based adjuvant for enhancing effector and memory CD8 T-cell responses. Immunology and cell biology 2014, 92 (4), 377-83.
12. Rhee, E. G.; Kelley, R. P.; Agarwal, I.; Lynch, D. M.; La Porte, A.; Simmons, N. L.; Clark, S. L.; Barouch, D. H., TLR4 ligands augment antigen-specific CD8+ T lymphocyte responses elicited by a viral vaccine vector. Journal of virology 2010, 84 (19), 10413-9.
13. Wiedemann, G. M.; Jacobi, S. J.; Chaloupka, M.; Krachan, A.; Hamm, S.; Strobl, S.; Baumgartner, R.; Rothenfusser, S.; Duewell, P.; Endres, S.; Kobold, S., A novel TLR7 agonist reverses NK cell anergy and cures RMA-S lymphoma-bearing mice. Oncoimmunology 2016, 5 (7), e1189051.
14. Cheadle, E. J.; Lipowska-Bhalla., G.; Dovedi, S. J.; Fagnano, E.; Klein, C.; Honeychurch, J.; Illidge, T. M., A TLR7 agonist enhances the antitumor efficacy of obinutuzumab in murine lymphoma models via NK cells and CD4 T cells. Leukemia 2017, 31 (10), 2278.
15. Hengge, U. R.; Ruzicka, T., Topical immunomodulation in dermatology: potential of toll-like receptor agonists. Dermatol Surg 2004, 30 (8), 1101-12.
16. Kaczanowska, S.; Joseph, A. M.; Davila, E., TLR agonists: our best frenemy in cancer immunotherapy. Journal of leukocyte biology 2013, 93 (6), 847-63.
17. Bandara, N. A.; Hansen, M. J.; Low, P. S., Effect of receptor occupancy on folate receptor internalization. Molecular pharmaceutics 2014, 11 (3), 1007-13.
18. Kniess, T.; Laube, M.; Wust, F.; Pietzsch, J., Technetium-99m based small molecule radiopharmaceuticals and radiotracers targeting inflammation and infection. Dalton transactions 2017, 46 (42), 14435-14451.
19. Kularatne, S. A.; Zhou, Z.; Yang, J.; Post, C. B.; Low, P. S., Design, synthesis, and preclinical evaluation of prostate-specific membrane antigen targeted (99m)Tc-radioimaging agents. Molecular pharmaceutics 2009, 6 (3), 790-800.
20. Henne, W. A.; Rothenbuhler, R.; Ayala-Lopez, W.; Kia, W.; Varghese, B.; Low, P. S., Imaging sites of infection using a 99mTc-labeled folate conjugate targeted to folate receptor positive macrophages. Molecular pharmaceutics 2012, 9 (5), 1435-40.
21. Kahan, S. M.; Wherry, E. J.; Zajac, A. J., T cell exhaustion during persistent viral infections. Virology 2015, 479-480, 180-93.
22. Stefanova, I.; Hemmer, B.; Vergelli, M.; Martin, R.; Biddison, W. E.; Germain, R. N., TCR ligand discrimination is enforced by competing ERK positive and SHP-1 negative feedback pathways. Nature immunology 2003, 4 (3), 248-54.
23. Lorenz, U., SHP-1 and SHP-2 in T cells: two phosphatases functioning at many Immunological reviews 2009, 228 (1), 342-59.
24. Watson, H. A.; Wehenkel, S.; Matthews, J.; Ager, A., SHP-1: the next checkpoint target for cancer immunotherapy? Biochemical Society transactions 2016, 44 (2), 356-62.
25. Hebeisen, M.; Baitsch, L.; Presotto, D.; Baumgaertner, P.; Romero, P.; Michielin, O.; Speiser, D. E.; Rufer, N., SHP-1 phosphatase activity counteracts increased T cell receptor affinity. The Journal of clinical investigation 2013, 123 (3), 1044-56.
26. Wiede, F.; Shields, B. J.; Chew, S. H.; Kyparissoudis, K.; van Vliet, C.; Galic, S.; Tremblay, M. L.; Russell, S. M.; Godfrey, D. I.; Tiganis, T., T cell protein tyrosine phosphatase attenuates T cell signaling to maintain tolerance in mice. The Journal of clinical investigation 2011, 121 (12), 4758-74.
27. Pike, K. A.; Hatzihristidis, T.; Bussieres-Marmen, S.; Robert, F.; Desai, N.; Miranda-Saavedra, D.; Pelletier, J.; Tremblay, M. L., TC-PTP regulates the IL-7 transcriptional response during murine early T cell development. Scientific reports 2017, 7 (1), 13275.
28. Wiedemann, G. M.; Jacobi, S. J.; Chaloupka, M.; Krachan, A.; Hamm, S.; Strobl, S.; Baumgartner, R.; Rothenfusser, S.; Duewell, P.; Endres, S.; Kobold, S., A novel TLR7 agonist reverses NK cell allergy and cures RMA-S lymphoma-bearing mice. Oncoimmunology 2016, 5 (7), e1189051.
29. Caron, G.; Duluc, D.; Fremaux, I.; Jeannin, P.; David, C.; Gascan, H.; Delneste, Y., Direct stimulation of human T cells via TLR5 and TLR7/8: flagellin and R-848 up-regulate proliferation and IFN-gamma production by memory CD4+ cells. Journal of immunology 2005, 175 (3), 1551-7.
30. Wille-Reece, U.; Flynn, B. J.; Lore, K.; Koup, R. A.; Miles, A. P.; Saul, A.; Kedl, R. M.; Mattapallil, J. J.; Weiss, W. R.; Roederer, M.; Seder, R. A., Toll-like receptor agonists influence the magnitude and quality of memory T cell responses after prime-boost immunization in nonhuman primates. The Journal of experimental medicine 2006, 203 (5), 1249-58.
31. Zarember, K. A.; Godowski, P. J., Tissue expression of human Toll-like receptors and differential regulation of Toll-like receptor mRNAs in leukocytes in response to microbes, their products, and cytokines. Journal of immunology 2002, 168 (2), 554-61.
32. Hornung, V.; Rothenfusser, S.; Britsch, S.; Krug, A.; Jahrsdorfer, B.; Giese, T.; Endres, S.; Hartmann, G., Quantitative expression of toll-like receptor 1-10 mRNA in cellular subsets of human peripheral blood mononuclear cells and sensitivity to CpG oligodeoxynucleotides. Journal of immunology 2002, 168 (9), 4531-7.
33. Strominger, N. L.; Brady, R.; Gullikson, G.; Carpenter, D. O., Imiquimod-elicited emesis is mediated by the area postrema, but not by direct neuronal activation. Brain Res Bull 2001, 55 (3), 445-51.
34. Harrison, L. I.; Astry, C.; Kumar, S.; Yunis, C., Pharmacokinetics of 852A, an imidazoquinoline Toll-like receptor 7-specific agonist, following intravenous, subcutaneous, and oral administrations in humans. Journal of clinical pharmacology 2007, 47 (8), 962-9.
35. Dudek, A. Z.; Yunis, C.; Harrison, L. I.; Kumar, S.; Hawkinson, R.; Cooley, S.; Vasilakos, J. P.; Gorski, K. S.; Miller, J. S., First in human phase I trial of 852A, a novel systemic toll-like receptor 7 agonist, to activate innate immune responses in patients with advanced cancer. Clinical cancer research: an official journal of the American Association for Cancer Research 2007, 13 (23), 7119-25.
36. Dummer, R.; Hauschild, A.; Becker, J. C.; Grob, J. J.; Schadendorf, D.; Tebbs, V.; Skalsky, J.; Kaehler, K. C.; Moosbauer, S.; Clark, R.; Meng, T. C.; Urosevic, M., An exploratory study of systemic administration of the toll-like receptor-7 agonist 852A in patients with refractory metastatic melanoma. Clinical cancer research: an official journal of the American Association or Cancer Research 2008, 14 (3), 856-64.
37. Perkins, H; Khodai, T.; Mechiche, Colman, P.; Burden, F.; Laxton, C.; Horscroft, N.; Corey, T.; Rodrigues, D.; Rawal, J.; Heyen, J.; Fidock, M.; Westby, M.; Bright, H., Therapy with TLR7 agonists induces lymphopenia: correlating pharmacology to mechanism in a mouse model. J Clin Immunol 2012, 32 (5), 1082-92.
38. Hasham, M. G.; Baxan, N.; Stuckey, D. J.; Branca, J.; Perkins, B.; Dent, O.; Duffy, T.; Hameed, T. S.; Stella, S. E.; Bellahcene, M.; Schneider, M. D.; Harding, S. E.; Rosenthal, N.; Sattler, S., Systemic autoimmunity induced by the TLR7/8 agonist Resiquimod causes myocarditis and dilated cardiomyopathy in a new mouse model of autoimmune heart disease. Dis Model Mech 2017, 10 (3), 259-270.
39. Katie, J.; David, N., The discovery of a novel prototype small molecule TLR7 agonist for the treatment of hepatitis C virus infection. MedChemComm 2011, 2 (3), 185-189.
40. Jones, P.; Pryde, D. C.; Tran, T. D.; Adam, F. M.; Bish, G.; Cabo, F.; Ciaramella, G.; Dixon, R.; Duckworth, J.; Fox, D. N.; Hay, D. A.; Hitchin, J.; Horscroft, N.; Howard, M.; Laxton, C.; Parkinson, T.; Parsons, G.; Proctor, K.; Smith, M. C.; Smith, N.; Thomas, A., Discovery of a highly potent series of TLR7 agonists. Bioorganic & medicinal chemistry letters 2011, 21 (19), 5939-43.
41. Hemmi, H.; Kaisho, T.; Takeuchi, O.; Sato, S.; Sanjo, H.; Hoshino, K.; Horiuchi, T.; Tomizawa, H.; Takeda, K.; Akira, S., Small anti-viral compounds activate immune cells via the TLR7 MyD88-dependent signaling pathway. Nature immunology 2002, 3 (2), 196-200.
42. Iversen, L. F.; Moller, K. B.; Pedersen, A. K.; Peters, G. H.; Petersen, A. S.; Andersen, H. S.; Branner, S.; Mortensen, S. B.; Moller, N. P., Structure determination of T cell protein-tyrosine phosphatase. The Journal of biological chemistry 2002, 277 (22), 19982-90.
43. Wilson, D. P.; Wan, Z. K.; Xu, W. X.; Kirincich, S. J.; Follows, B. C.; Joseph-McCarthy, D.; Foreman, K.; Moretto, A.; Wu, J.; Zhu, M.; Binnun, E.; Zhang, Y. L.; Tam, M.; Erbe, D. V.; Tobin, J.; Xu, X.; Leung, L.; Shilling, A.; Tam, S. Y.; Mansour, T. S.; Lee, J., Structure-based optimization of protein tyrosine phosphatase 1B inhibitors: from the active site to the second phosphotyrosine binding site. Journal of medicinal chemistry 2007, 50 (19), 4681-98.
44. Ignacio, B. J.; Albin, T. J.; Esser-Kahn, A. P.; Verdoes, M., Toll-like Receptor Agonist Conjugation; A Chemical Perspective. Bioconjugate chemistry 2018, 29 (3), 587-603.
45. Parente-Pereira, A. C.; Burnet, J.; Ellison, D.; Foster, J.; Davies, D. M.; van der Stegen, S.; Burbridge, S.; Chiapero-Stanke, L.; Wilkie, S.; Mather, S.; Maher, J., Trafficking of CAR-engineered human T cells following regional or systemic adoptive transfer in SCID beige mice. J Clin Immunol 2011, 31 (4), 710-8.
46. Dobrenkov, K.; Olszewska, M.; Likar, Y.; Shenker, L.; Gunset, G.; Cai, S.; Pillarsetty, N.; Hricak, H.; Sadelain, M.; Ponomarev, V., Monitoring the efficacy of adoptively transferred prostate cancer-targeted human T lymphocytes with PET and bioluminescence imaging. Journal of nuclear medicine: official publication, Society of Nuclear Medicine 2008, 49 (7), 1162-70.
47. Emami-Shahri, N.; Foster, J.; Kashani, R.; Gazinska, P.; Cook, C.; Sosabowski, J.; Maher, I.; Papa, S., Clinically compliant spatial and temporal imaging: of chimeric antigen receptor T-cells. Nature communications 2018, 9 (1), 1081.
48. Bruno, R.; Giannasio, P.; Ronga, Baudin, E.; Travagli, J. P.; Russo, D.; Filetti, S.; Schlumberger, M., Sodium iodide symporter expression and radioiodine distribution in extrathyroidal tissues. J Endocrinol Invest 2004, 27 (11), 1010-4.
49. Vedvyas, Y.; Shevlin, E.; Zamam, M.; Min, I. M.; Amor-Coarasa, A.; Park, S.; Park, S.; Kwon, K. W.; Smith, T.; Luo, Y.; Kim, D.; Kim, Y.; Law, B.; Ting, R.; Babich, J.; Jin, M. M., Longitudinal PET imaging demonstrates biphasic CAR T cell responses in survivors. JCI insight 2016, 1 (19), e90064.
50. Zhang, H.; Moroz, M. A.; Serganova, I.; Ku, T.; Huang, R.; Vider, J,; Maecke, H. R.; Larson, S. M.; Blasberg, R.; Smith-Jones, P. M., Imaging expression of the human somatostatin receptor subtype-2 reporter gene with 68Ga-DOTATOC. Journal of nuclear medicine: official publication, Society of Nuclear Medicine 2011, 52 (1), 123-31.
51. Bhattacharyya, S.; Dixit, M., Metallic radionuclides in the development of diagnostic and therapeutic radiopharmaceuticals. Dalton transactions 2011, 40 (23), 6112-28.
52. Khan, O.; Giles, J. R.; McDonald, S.; Mamie, S.; Ngiow, S. F.: Patel, K. P.; Werner, M. T.; Huang, A. C.; Alexander, K. A.; Wu, J. E.; Attanasio, J.; Yan, P.; George, S. M,; Bengsch, B.; Staupe, R. P.; Donahue, G.; Xu, W.; Amaravadi, R. K.; Xu, X.; Karakousis, G. C.; Mitchell, T. C.; Schuchter, L. M.; Kaye, J.; Berger, S. L.; Wherry, E, J., TOX transcriptionally and epigenetically programs CD8(+) T cell exhaustion. Nature 2019.
53. Alfei, F.; Kanev, K.; Hofmann, M.; Wu, M.; Ghoneim, H. E.; Roelli, P.; Utzschneider, D. T.; von Hoesslin, M.; Cullen, J. G.; Fan, Y.; Eisenberg, V.; Wohlleber, D.; Steiger, K.; Merkler, D.; Delorenzi, M.; Knolle, P. A.; Cohen, C. J.; Thimme, R.; Youngblood, B.; Zehn, D., TOX reinforces the phenotype and longevity of exhausted T cells in chronic viral infection. Nature 2019.
54. Kawai, T.; Akira, S., Signaling to NF-kappaB by Toll-like receptors. Trends in molecular medicine 2007, 13 (11), 460-9.
55. Chen, J.; Lopez-Moyado, I. F.; Seo, H.; Lio, C. J.; Hempleman, L. J.; Sekiya, T.; Yoshimura, A.; Scott-Browne, J. P.; Rao, A., NR4A transcription factors limit CAR T cell function in solid tumours. Nature 2019, 567 (7749), 530-534.
56. Larkin, B.; Ilyukha, V.; Sorokin, M.; Buzdin, A.; Vannier, E.; Poltorak, A., Cutting Edge: Activation of STING in T Cells Induces Type 1 IFN Responses and Cell Death. Journal of immunology 2017, 199 (2), 397-402.
57. Prinz, P. U.; Mendler, A. N.; Masouris, I.; Durner, L.; Oberneder, R.; Noessner, E., High DGK-alpha and disabled MAPK pathways cause dysfunction of human tumor-infiltrating CD8+ T cells that is reversible by pharmacologic intervention. Journal of immunology 2012, 188 (12), 5990-6000.
58. Gorelik, L.; Flavell, R. A., Immune-mediated eradication of tumors through the blockade of transforming growth factor-β signaling in T cells. Nature medicine 2001, 7 (10), 1118.
59. Herbertz, S.; Sawyer, J. S.; Stauber, A. J.; Gueorguieva, I.; Driscoll, K. E.; Estrem, S. T.; Cleverly, A. L.; Desaiah, D.; Guba, S. C.; Benhadji, K. A.; Slapak, C. A.; Lahn, M. M., Clinical development of galunisertib (LY2157299 monohydrate), a small molecule inhibitor of transforming growth factor-beta signaling pathway. Drug design, development and therapy 2015, 9, 4479-99,
60. Jin, C. H.; Krishnaiah, M.; Sreenu, D.; Subrahmanyam, V. B.; Rao, K. S.; Lee, H. J.; Park, S. J.; Park, H. J.; Lee, K.; Sheen, Y. Y.; Kim, D. K., Discovery of N-((4-([1,2,4]triazolo[1,5-a]pyridin-6-yl)-5-(6-methylpyridin-2-yl)-1H-imidazol-2-yl)methyl)-2-fluoroaniline (EW-7197): a highly potent, selective, and orally bioavailable inhibitor of TGF-beta type 1 receptor kinase as cancer immunotherapeutic/antifibrotic agent. Journal of medicinal chemistry 2014, 57 (10), 4213-38.
61. Wang, D.; Quiros, J.; Mahuron, K.; Pai, C. C.; Ranzani, V.; Young, A.; Silveria, S.; Hardin, T.; Abnousian, A.; Pagani, M.; Rosenblum, M. D.; Van Gool, F.; Fong, L.; Bluestone, J. A.; DuPage, M., Targeting EZH2 Reprograms Intratumoral Regulators T Cells to Enhance Cancer Immunity. Cell reports 2018, 23 (11), 3262-3274

What is claimed is:

1. A system to rejuvenate an exhausted classical CAR T cell comprising at least two components: a first component is a conjugate comprising a targeting ligand covalently linked to a payload drug; and a second component is a targeting ligand binding module linked to a membrane-anchoring module, wherein the targeting ligand binding module of the second component recognizes the targeting ligand in the first component with high affinity to form a complex, the payload drug either blocks the inhibitory signaling of the exhausted classical CAR T cell or re-activates said CAR T cell through an antigen-independent pathway, and the membrane-anchoring module mediates internalization of the at least two components complex into the exhausted classical CAR T cell.

2. The system according to claim 1, wherein the targeting ligand of the first component is fluorescein isothiocyanate (FITC) or FK506.

3. The system according to claim 1, wherein the targeting ligand binding module of the second component comprises an anti-FITC antibody fragment or a FK506-binding protein (FKBP).

4. The system according to claim 1, wherein the membrane-anchoring module is a foliate receptor.

5. The system according to claim 1, wherein the first component comprises a releasable linker between the targeting ligand and the payload drug.

6. The system according to claim 1, wherein the first component comprises a non-releasable linker between the targeting ligand and the payload drug.

7. The system according to claim 1, wherein the binding affinity between the targeting ligand and the ligand-binding module is in a sub-nanomolar range.

8. The system according to claim 1, wherein the payload of drug is a toll-like receptor 7 (TLR7) agonist or a simulator of interferon genes (STING) agonist.

9. The system according to claim 1, wherein the payload of drug is an inhibitor of Src-homology region 2 domain-containing phosphatase 1 (SHP1), Src-homology region 2 domain-containing phosphatase 2 (SHP2), T cell protein tyrosine phosphatase (TC-PTP), diacylglycerol kinase alpha (DGKα), or transforming growth factor beta (TGFβ).

10. The system according to claim 8, wherein the TLR7 agonist has the structure of

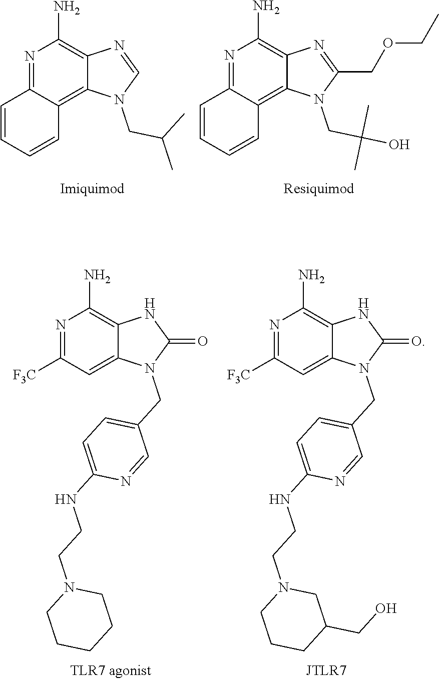

11. The system according to claim 1, wherein the first component is a fluorescein-TLR7 agonist having the structure of

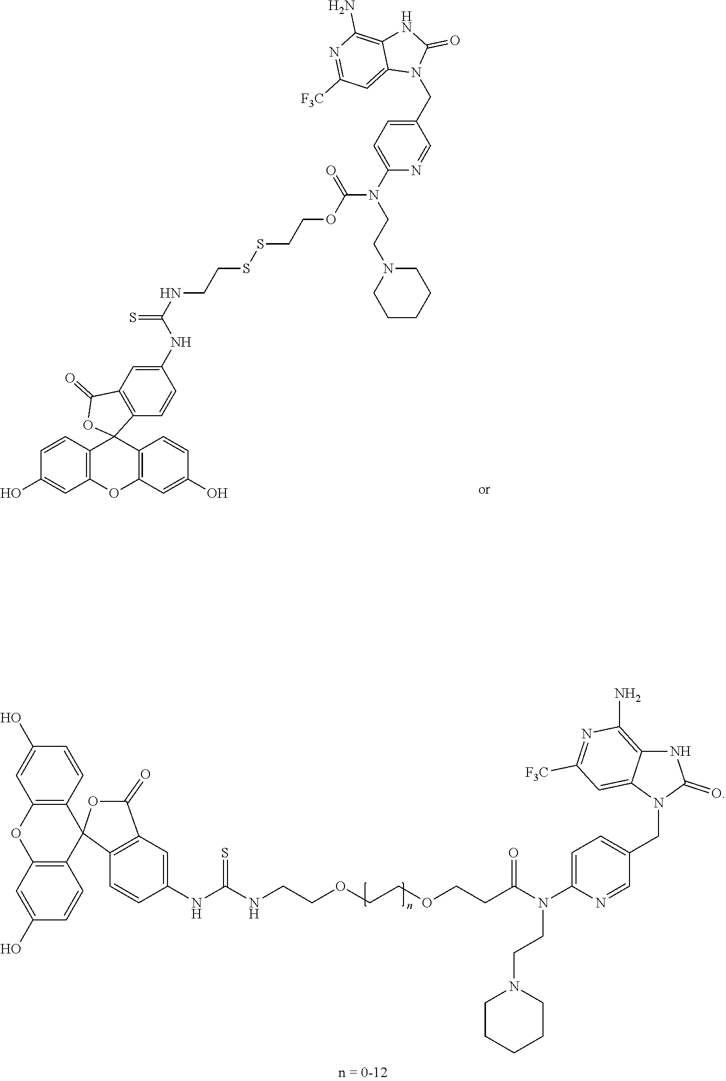

12. The system according to claim 1, wherein the first component is a FK506-TLR7 agonist having the structure of

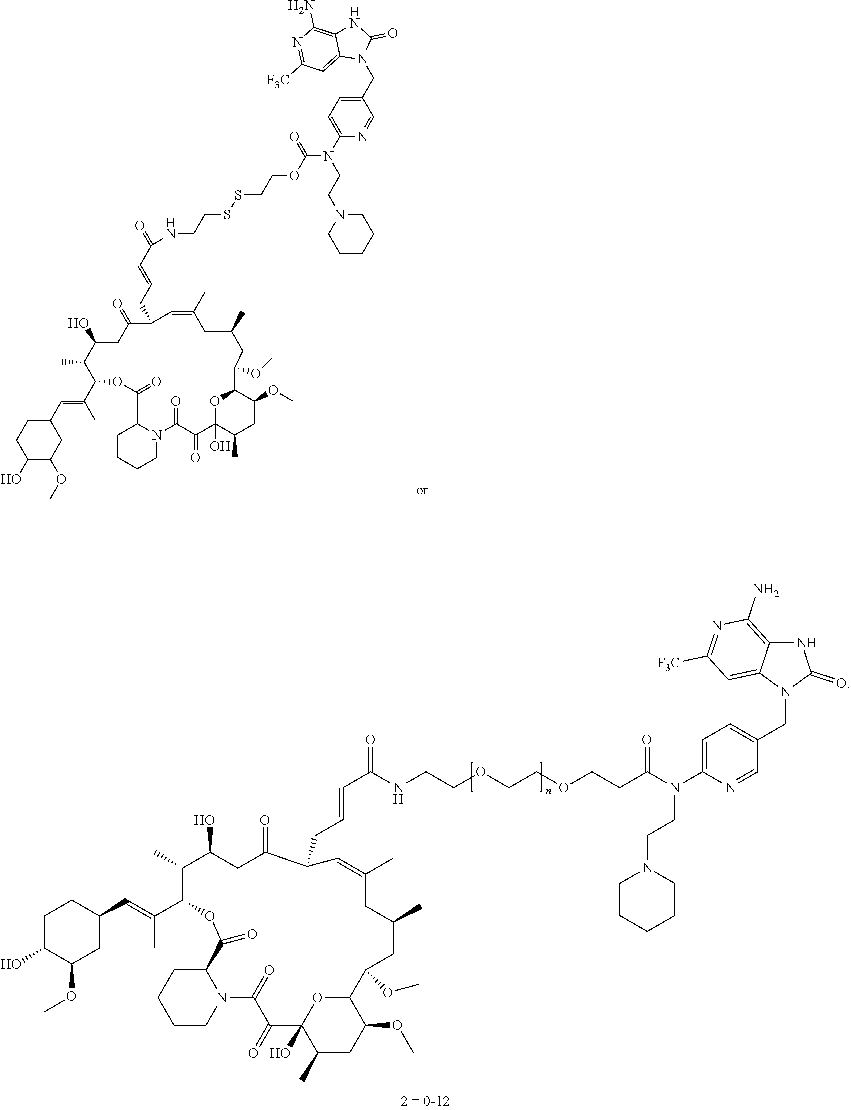

13. The system according to claim 1, wherein the first component is one of the following:

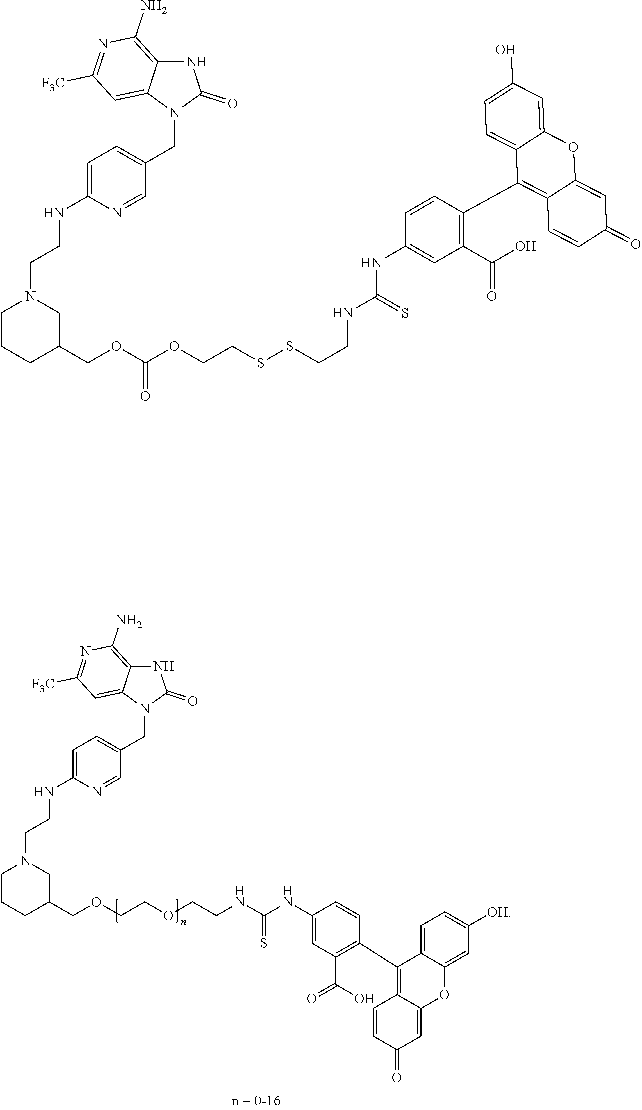

14. The system according to claim 1, wherein the first component comprising the payload drug is selected from the group consisting of the following TC-PTP inhibitors:

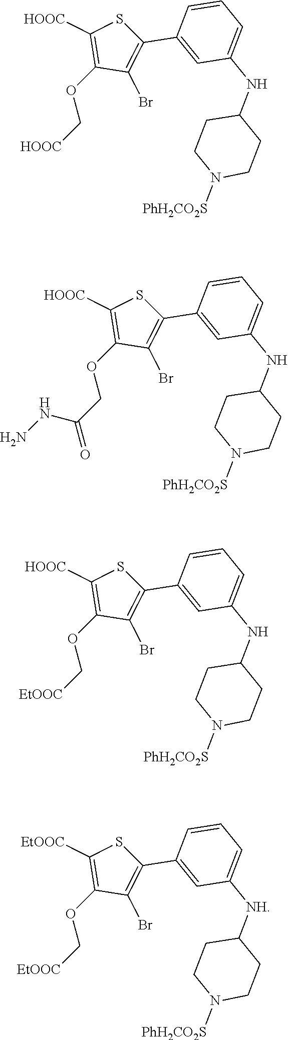

15. The system according to claim 14, wherein the TC-PTP inhibitor is connected to the fluorescein or FK506 (tacrolimus) to form the following structures:

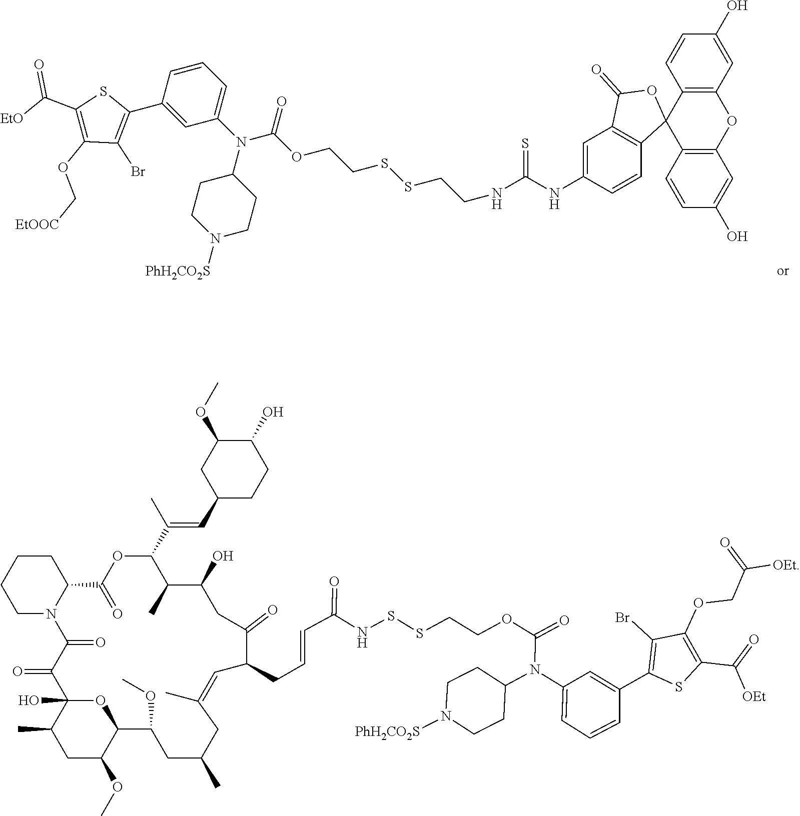

16. The system according to claim 1, wherein the first component comprises a spacer between the targeting ligand and the payload drug selected from the group consisting of the following structures:

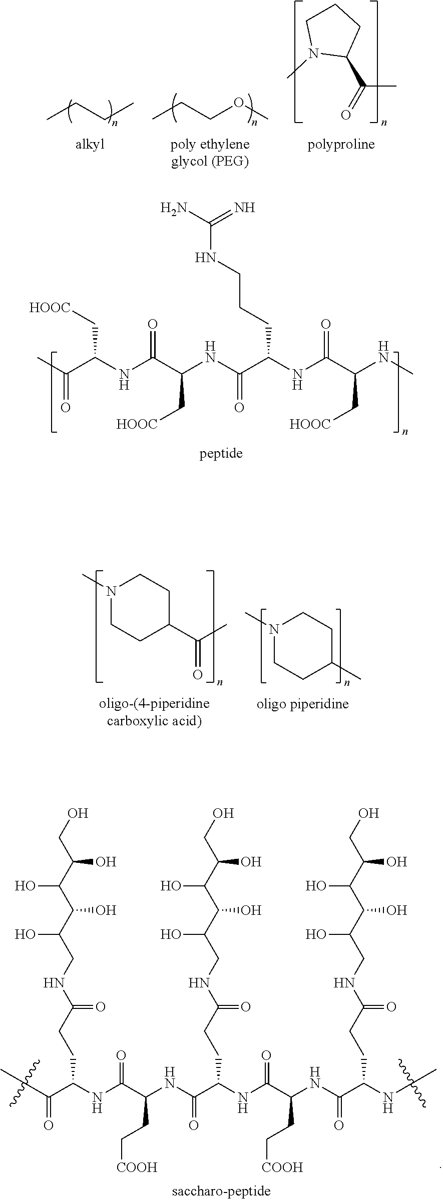

17. A method to rejuvenate an exhausted CAR T cell, comprising:
a. providing said exhausted CAR T cell a first component comprising a conjugate, wherein the conjugate comprises a targeting ligand covalently linked to a payload of drug through a releasable linker or a non-releasable linker;
b. providing said exhausted CAR T cell a second component comprising a fusion receptor, wherein the fusion receptor comprises a targeting ligand binding module and a membrane-bound receptor module;
c. letting the targeting ligand binding module of the second component bind to the targeting ligand in the first component to form a complex,
d. letting the membrane-bound receptor module mediate internalization of the complex into the exhausted CAR T cell; and
e. letting the payload drug either block the inhibitory signaling of the exhausted CAR T cell or re-activate said CAR T cell through an antigen-independent pathway.

18. The method according to claim 17, wherein the payload drug executes its function within the endosome of the exhausted CAR T cell, and the targeting ligand and the payload drug are linked by a non-releasable linker.

19. The method according to claim 17, wherein the payload drug executes its function as a free drug in the cytosol of the exhausted CAR. T cell, and the targeting ligand and the payload drug are linked by a releasable linker.

20. The method according to claim 17, wherein the targeting ligand of the first component is fluorescein isothiocyanate (FITC) or FK506.

21. The method according to claim 17, wherein the targeting ligand binding module of the second component is an anti-FITC antibody fragment or a FK506-binding protein (FKBP).

22. The method according to claim 17, wherein the payload of drug is a toll-like receptor 7 (TLR7) agonist or a simulator of interferon genes (STING) agonist.

23. The method according to claim 17, wherein the payload of drug is an inhibitor of Src-homology region 2 domain-containing phosphatase 1 (SHP1), Src-homology region 2 domain-containing phosphatase 2 (SHP2), T cell protein tyrosine phosphatase (TC-PTP), diacylglycerol kinase alpha (DGKα), or transforming growth factor beta (TGFβ).

24. The method according to claim 17, wherein the TLR7 agonist has the structure of

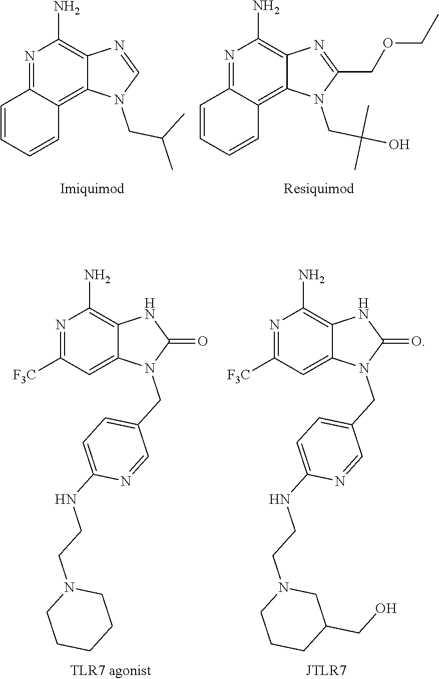

25. The method according to claim 17, wherein the first component is a fluorescein-TLR7 agonist having the structure of

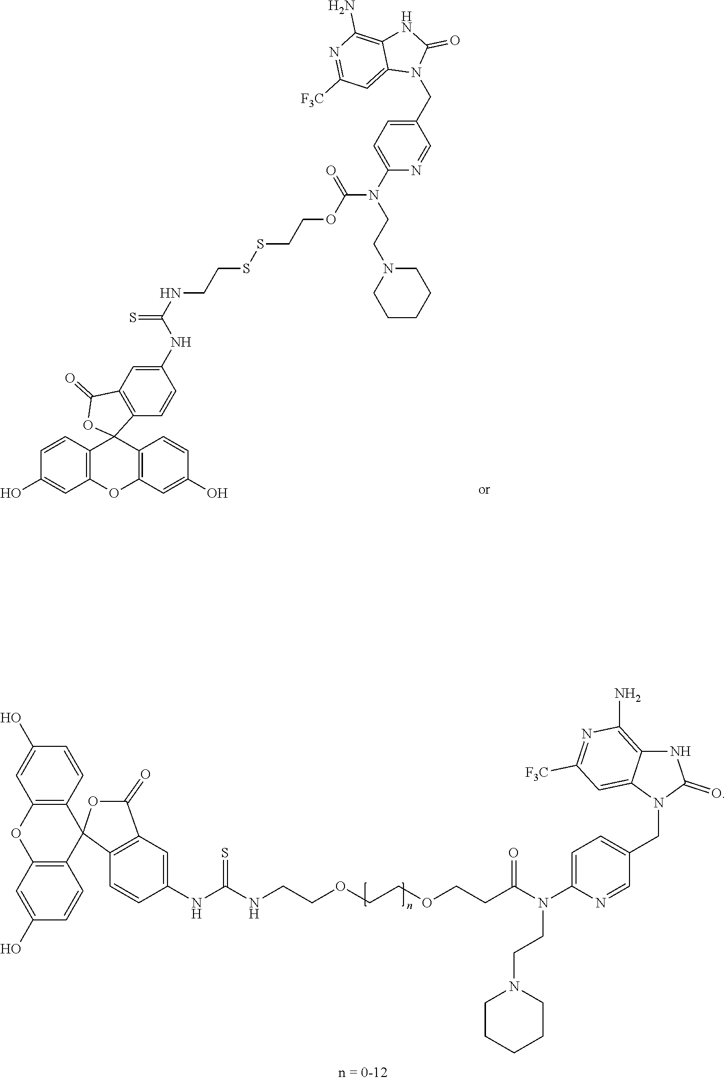

26. The method according to claim 17, wherein the first component is a FX506-TLR7 agonist having the structure of

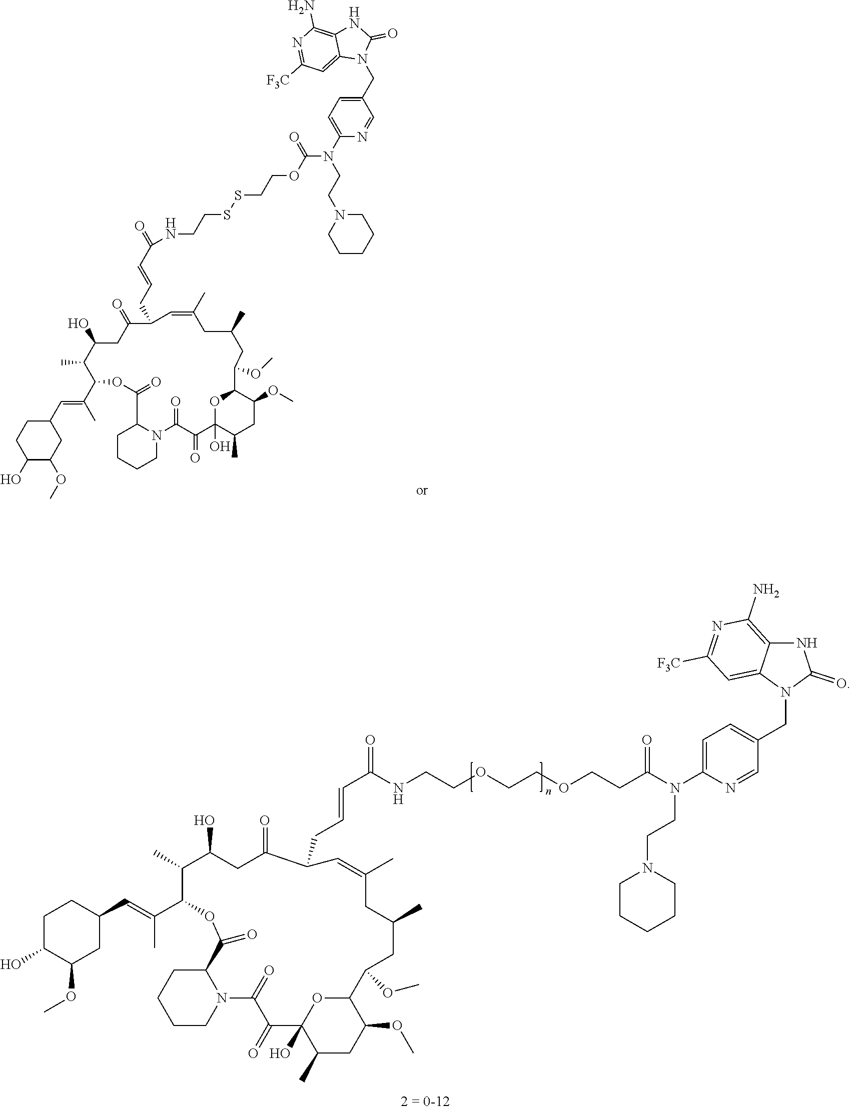

27. The method according to claim 17, wherein the first component is one of the following

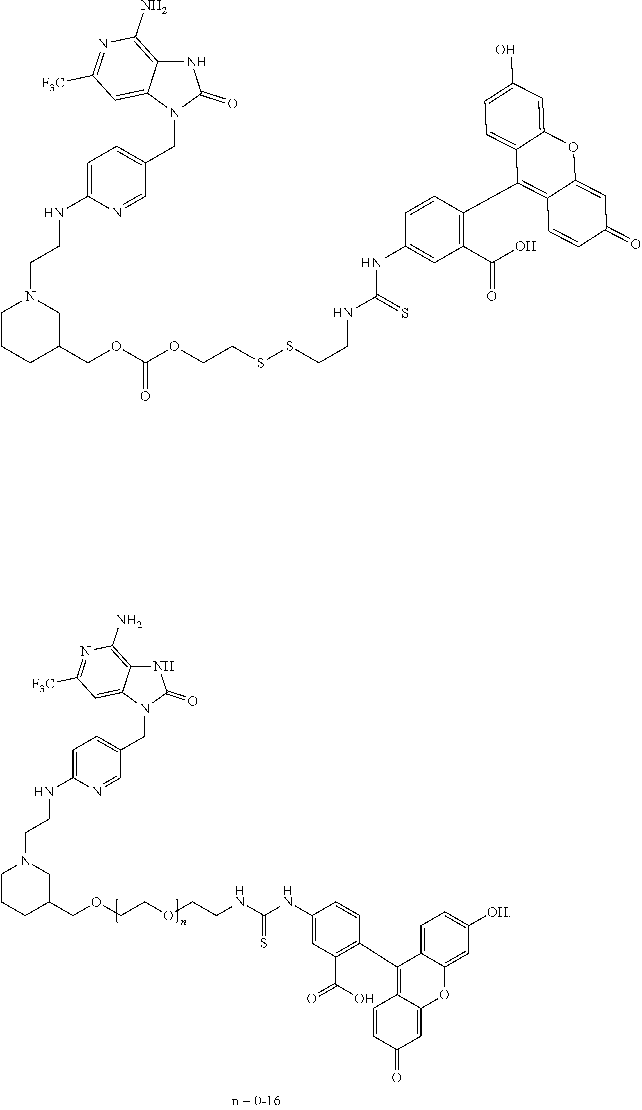

28. The method according to claim 17, wherein the first component comprising the payload drug selected from the group consisting of following TC-PTP inhibitors:

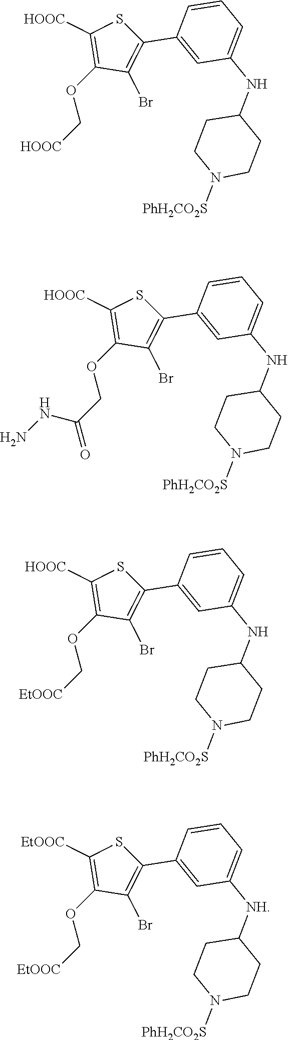

29. The method according to claim 28, wherein the TC-PTP inhibitor is connected to the fluorescein or FK506 (tacrolimus) to form the following structures:

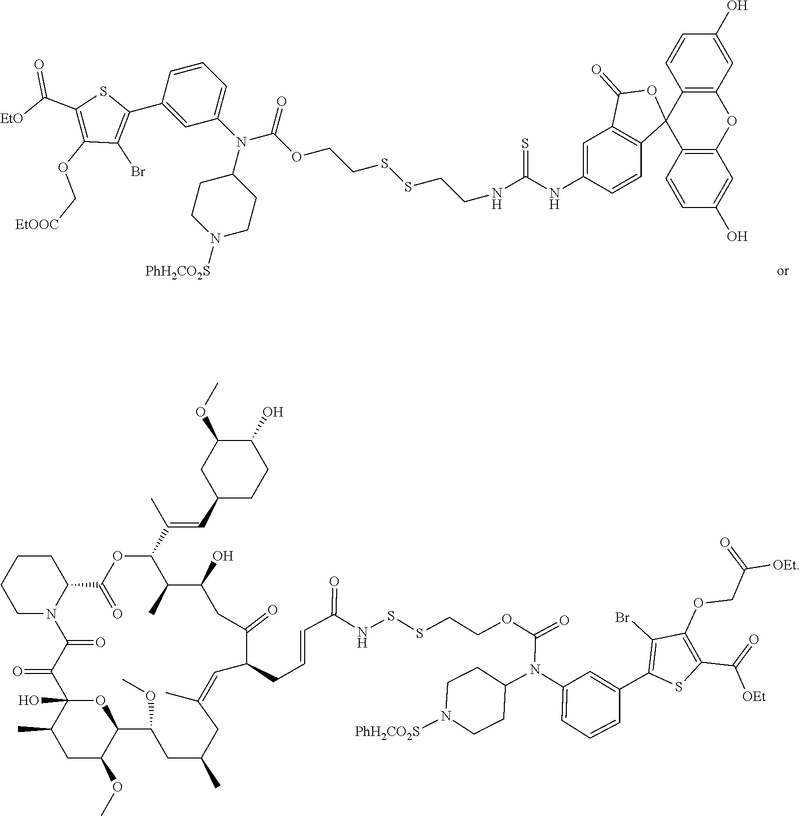

30. The method according to claim 17, wherein the first component comprising a spacer between the targeting ligand and the payload drug is selected from the group consisting of the following structures: [chemical structure]
